(19) United States
(12) Patent Application Publication
(10) Pub. No.: US 2017/0273663 A1
(43) Pub. Date: Sep. 28, 2017

(54) IMAGE PROCESSING FOR AN ULTRASONIC FETAL IMAGING DEVICE

(71) Applicant: Elwha LLC, Bellevue, WA (US)

(72) Inventors: Michael H. Baym, Cambridge, MA (US); Roderick A. Hyde, Redmond, WA (US); Muriel Y. Ishikawa, Livermore, CA (US)

(21) Appl. No.: 15/079,941

(22) Filed: Mar. 24, 2016

(51) Int. Cl. A61B 8/08; A61B 8/00

(52) U.S. Cl. CPC A61B 8/0866; A61B 8/5207; A61B 8/483; A61B 8/4483; A61B 8/5223; A61B 8/4209; A61B 8/485; A61B 8/429; A61B 8/54; A61B 8/565; A61B 8/466; A61B 5/11

(57) ABSTRACT

Devices and methods for fetal imaging are described. In some embodiments, a method of fetal imaging includes, but is not limited to, detecting at least one portion of a fetus within a body based upon one or more ultrasonic signals received from the body; identifying the at least one detected portion of the fetus; determining at least one non-detected portion of the fetus based upon the identification of the at least one detected portion of the fetus; and inferring an identification of the non-detected portion of the fetus based on the identification of the at least one detected portion of the fetus.

If an Application Data Sheet (ADS) has been filed on the filing date of this application, it is incorporated herein by reference. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO PRIORITY APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a method of fetal imaging includes, but is not limited to, detecting at least one portion of a fetus within a body based upon one or more ultrasonic signals received from the body; identifying the at least one detected portion of the fetus; determining at least one non-detected portion of the fetus based upon the identification of the at least one detected portion of the fetus; and inferring an identification of the non-detected portion of the fetus based on the identification of the at least one detected portion of the fetus.

In an aspect, a non-transitory computer readable medium includes one or more executable instructions stored thereon. The one or more executable instructions include, but are not limited to, one or more instructions that cause at least one computer processor executing the one or more instructions to: detect at least one portion of a fetus within a body based upon one or more ultrasonic signals received from the body; identify the at least one detected portion of the fetus; determine at least one non-detected portion of the fetus based upon the identification of the at least one detected portion of the fetus; and infer an identification of the non-detected portion of the fetus based on the identification of the at least one detected portion of the fetus.

In an aspect, a method of providing a connectivity model for an ultrasonically imaged fetus includes, but is not limited to, identifying one or more portions of a fetus within a body based upon one or more ultrasonic signals received from the body; and generating a connectivity model for the fetus based upon the one or more identified portions of the fetus.

In an aspect, a method of providing a textured image of an ultrasonically imaged fetus includes, but is not limited to, identifying one or more portions of a fetus within a body based upon one or more ultrasonic signals received from the body; and applying a texture map to a graphical rendering of the one or more identified portions of the fetus to generate a textured image of the fetus.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a fetal imaging device.

FIG. 2 is a block diagram of an embodiment of an ultrasound element of a fetal imaging device such as shown in FIG. 1.

FIG. 3 is a block diagram of an embodiment of a fetal imaging device such as shown in FIG. 1.

FIG. 4 is an illustration of an embodiment of a fetal imaging device such as shown in FIG. 1 and a body bearing a fetus.

FIG. 5 is an illustration of an embodiment of a fetal imaging device such as shown in FIG. 1 and a body bearing a fetus.

FIG. 6 is an illustration of an embodiment of a fetal imaging device such as shown in FIG. 1.

FIG. 7 is an illustration of an embodiment of an ultrasound element of a fetal imaging device such as shown in FIG. 1 configured to apply one or more ultrasonic signals to one or more portions of a body bearing a fetus and further configured to receive one or more ultrasonic signals associated with one or more shear waves transmitted through the one or more portions of the body bearing the fetus as a result of the one or more applied ultrasonic signals.

FIG. 8 is an illustration of an embodiment of a first ultrasound element of a fetal imaging device such as shown in FIG. 1 configured to apply one or more ultrasonic signals to one or more portions of a body bearing a fetus and a second ultrasound element of a fetal imaging device such as shown in FIG. 1 configured to receive one or more ultrasonic signals associated with one or more shear waves transmitted through the one or more portions of the body bearing the fetus as a result of the one or more applied ultrasonic signals.

FIG. 9 is a block diagram of an embodiment of a fetal imaging device such as shown in FIG. 1.

FIG. 10 is an illustration of an embodiment of a fetal imaging device such as shown in FIG. 1.

FIG. 11 is a block diagram of an embodiment of a fetal imaging device such as shown in FIG. 1.

FIG. 12 is a block diagram of an embodiment of a fetal imaging device such as shown in FIG. 1.

FIG. 13 is a block diagram of an embodiment of a fetal imaging device such as shown in FIG. 1.

FIG. 14 is a block diagram of an embodiment of a fetal imaging device such as shown in FIG. 1.

FIG. 15 is a block diagram of an embodiment of a fetal imaging device such as shown in FIG. 1 and a remote device in communication with the fetal imaging device.

FIG. 16 is an illustration of an embodiment of an image of one or more identified portions of a fetus that are detected with a fetal imaging device such as shown in FIG. 1.

FIG. 17 is an illustration of an embodiment of a textured image of one or more identified portions of a fetus that are detected with a fetal imaging device such as shown in FIG. 1.

FIG. 18 is an illustration of an embodiment of an image of one or more identified portions of a fetus that are detected with a fetal imaging device such as shown in FIG. 1.

FIG. 19 is an illustration of an embodiment of an image of one or more identified portions of a fetus that are detected with a fetal imaging device such as shown in FIG. 1 and one or more non-detected portions of the fetus that are identified based upon the one or more detected portions of the fetus.

FIG. 20 is an illustration of an embodiment of an image of one or more identified portions of a first fetus and one or more identified portions of a second fetus that are detected with a fetal imaging device such as shown in FIG. 1 and one or more non-detected portions of the first fetus and one or more non-detected portions of the second fetus that are identified based upon at least one of the one or more detected portions of the first fetus or the one or more detected portions of the second fetus.

FIG. 21 is a flowchart illustrating a method of fetal imaging.

FIG. 22 is a flowchart illustrating one or more aspects of a method such as shown in FIG. 21.

FIG. 23 is a flowchart illustrating one or more aspects of a method such as shown in FIG. 21.

FIG. 24 is a flowchart illustrating one or more aspects of a method such as shown in FIG. 21.

FIG. 25 is a flowchart illustrating one or more aspects of a method such as shown in FIG. 21.

FIG. 26 is a flowchart illustrating one or more aspects of a method such as shown in FIG. 21.

FIG. 27 is a flowchart illustrating one or more aspects of a method such as shown in FIG. 21.

FIG. 28 is a flowchart illustrating one or more aspects of a method such as shown in FIG. 21.

FIG. 29 is a flowchart illustrating one or more aspects of a method such as shown in FIG. 21.

FIG. 30 is a flowchart illustrating one or more aspects of a method such as shown in FIG. 21.

FIG. 31 is a flowchart illustrating one or more aspects of a method such as shown in FIG. 21.

FIG. 32 is a flowchart illustrating one or more aspects of a method such as shown in FIG. 21.

FIG. 33 is a flowchart illustrating one or more aspects of a method such as shown in FIG. 21.

FIG. 34 is a flowchart illustrating one or more aspects of a method such as shown in FIG. 21.

FIG. 35 is a flowchart illustrating one or more aspects of a method such as shown in FIG. 21.

FIG. 36 is a flowchart illustrating one or more aspects of a method such as shown in FIG. 21.

FIG. 37 is a flowchart illustrating one or more aspects of a method such as shown in FIG. 21.

FIG. 38 is a flowchart illustrating one or more aspects of a method such as shown in FIG. 21.

FIG. 39 is a flowchart illustrating one or more aspects of a method such as shown in FIG. 21.

FIG. 40 is a flowchart illustrating one or more aspects of a method such as shown in FIG. 21.

FIG. 41 is a flowchart illustrating one or more aspects of a method such as shown in FIG. 21.

FIG. 42 is a flowchart illustrating one or more aspects of a method such as shown in FIG. 21.

FIG. 43 is a flowchart illustrating one or more aspects of a method such as shown in FIG. 21.

FIG. 44 is a flowchart illustrating one or more aspects of a method such as shown in FIG. 21.

FIG. 45 is a flowchart illustrating a method of inferring an identification of at least one non-detected portion of a fetus.

FIG. 46 is a flowchart illustrating one or more aspects of a method such as shown in FIG. 45.

FIG. 47 is a flowchart illustrating one or more aspects of a method such as shown in FIG. 45.

FIG. 48 is a flowchart illustrating one or more aspects of a method such as shown in FIG. 45.

FIG. 49 is a flowchart illustrating one or more aspects of a method such as shown in FIG. 45.

FIG. 50 is a flowchart illustrating one or more aspects of a method such as shown in FIG. 45.

FIG. 51 is a flowchart illustrating one or more aspects of a method such as shown in FIG. 45.

FIG. 52 is a flowchart illustrating one or more aspects of a method such as shown in FIG. 45.

FIG. 53 is a flowchart illustrating one or more aspects of a method such as shown in FIG. 45.

FIG. 54 is a flowchart illustrating one or more aspects of a method such as shown in FIG. 45.

FIG. 55 is a flowchart illustrating one or more aspects of a method such as shown in FIG. 45.

FIG. 56 is a flowchart illustrating one or more aspects of a method such as shown in FIG. 45.

FIG. 57 is a flowchart illustrating one or more aspects of a method such as shown in FIG. 45.

FIG. 58 is a flowchart illustrating one or more aspects of a method such as shown in FIG. 45.

FIG. 59 is a flowchart illustrating one or more aspects of a method such as shown in FIG. 45.

FIG. 60 is a flowchart illustrating a method of generating a connectivity model for a fetus.

FIG. 61 is a flowchart illustrating one or more aspects of a method such as shown in FIG. 60.

FIG. 62 is a flowchart illustrating one or more aspects of a method such as shown in FIG. 60.

FIG. 63 is a flowchart illustrating a method of generating a textured image of one or more identified portions a fetus.

FIG. 64 is a flowchart illustrating one or more aspects of a method such as shown in FIG. 63.

FIG. 65 is a flowchart illustrating one or more aspects of a method such as shown in FIG. 63.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented here.

Generally, a fetal imaging device can include one or more ultrasound elements. The one or more ultrasound elements can include at least one ultrasound source configured to apply one or more ultrasonic signals to a body and at least one ultrasound receiver to receive one or more ultrasonic signals from the body. By processing the one or more received ultrasonic signals, the fetal imaging device can identify or generate an image of one or more portions of a fetus within the body. For example, the fetal imaging device can include a controller configured to process electrical signals or digital values associated with the one or more received ultrasonic signals. The fetal imaging device can be used for a variety of fetal monitoring applications. For example, some applications include fetal monitoring by a medical professional, personal monitoring, and fetal monitoring for an animal by a pet owner, breeder, or veterinary care professional.

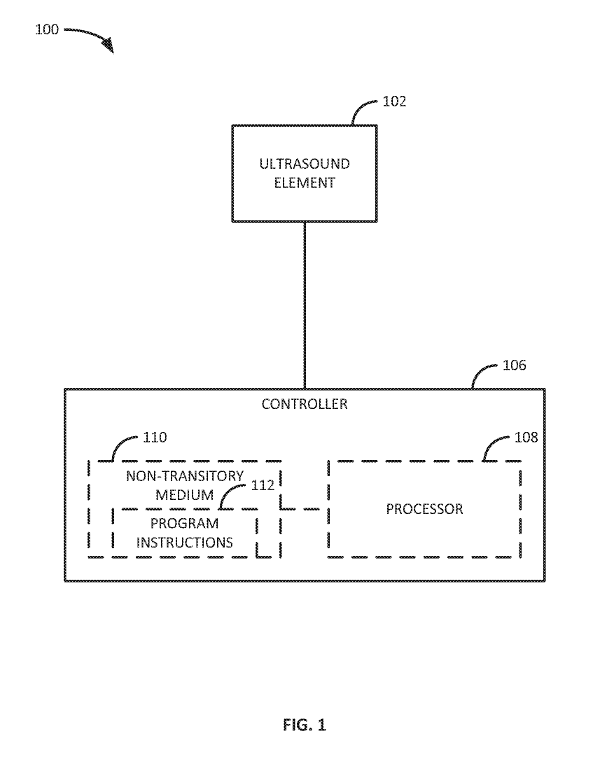

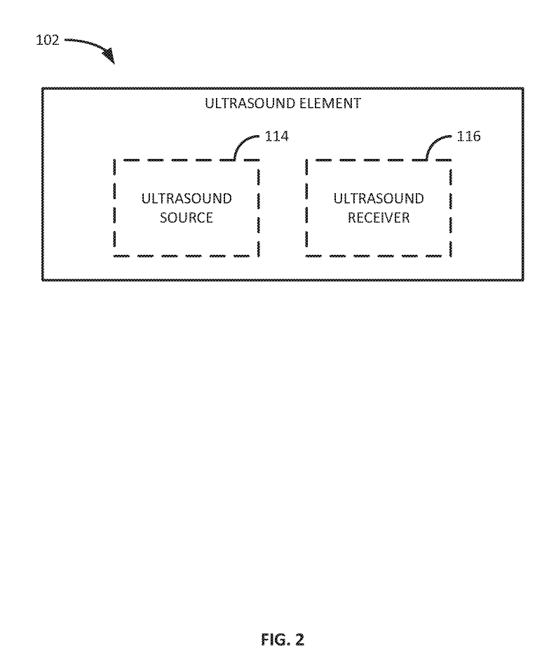

Referring to FIGS. 1-15, embodiments of a fetal imaging device 100 are shown to include at least one ultrasound element 102, such as an ultrasonic sensor, transceiver, transducer, or the like. As shown in FIG. 2, an ultrasound element 102 can include at least one of an ultrasound source 114 or an ultrasound receiver 116. For example, the ultrasound element 102 can include an ultrasonic sensor, transceiver, or transducer operable as the ultrasound source 114, the ultrasound receiver 116, or both of the ultrasound source 114 and the ultrasound receiver 116. The ultrasound source can deliver a compressive wave, also known as a p-wave, or it can deliver a shear wave, also known as a s-wave, or the source can deliver both ultrasound p-waves and s-waves.

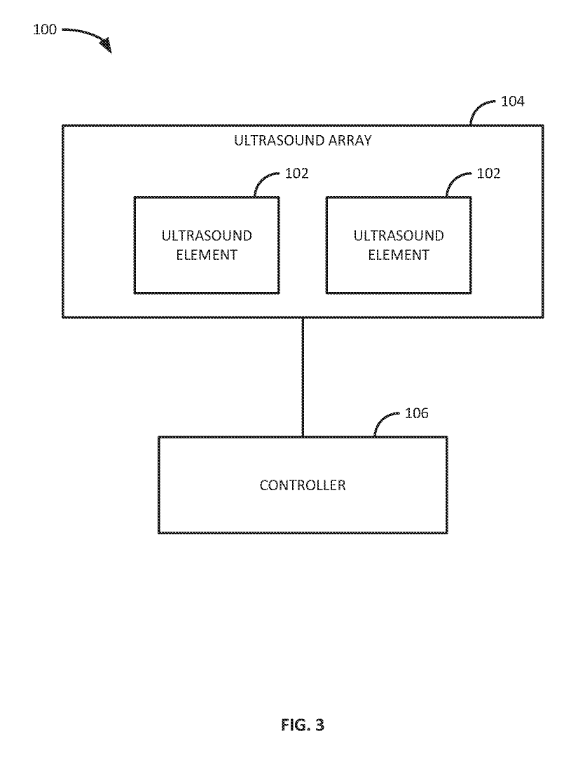

The fetal imaging device 100 can include a plurality of ultrasound elements 102. For example, as shown in FIG. 3, two or more ultrasound elements 102 can form an ultrasound array 104. The ultrasound elements 102 of the array 104 can be independently operable or configured to operate in concert with one another. For example, the array 104 can receive one or more control signals that affect a single ultrasound element 102 or multiple ultrasound elements 102 of the array 104. In some embodiments, at least one of the ultrasound elements 102 includes the ultrasound source 114 and the ultrasound receiver 116. For example, one or more of the ultrasound elements 102 of the array can include an ultrasonic sensor, transceiver, or transducer operable as an ultrasound source and an ultrasound receiver. In some embodiments, the ultrasound source 114 is included in a first ultrasound element 102, and the ultrasound receiver 116 is included in a second ultrasound element 102. For example, the first ultrasound element 102 can include an ultrasonic sensor, transceiver, or transducer operable as the ultrasound source 114, and the second ultrasound element 102 can include an ultrasonic sensor, transceiver, or transducer operable as the ultrasound receiver 116.

The fetal imaging device 100 can include or can be communicatively coupled to a controller 106. An embodiment of the controller 106 is shown in FIG. 1. The controller 106 can include a processor 108, such as a general-purpose processor, an application specific integrated circuit (ASIC), one or more field programmable gate arrays (FPGAs), a digital-signal-processor (DSP), a group of processors or processing cores, or other suitable electronic processing components. The processor 108 can be communicatively coupled to at least one non-transitory medium 110 (e.g., RAM, ROM, Flash Memory, hard disk storage, or the like) for storing data and program instructions 112 that enable the controller 106 to perform various operations described herein when executed by the processor 108. The non-transitory medium 110 can include non-transient volatile memory or non-volatile memory. The non-transitory medium 110 can include database components, object code components, script components, or any other type of information structure for supporting the various activities and information structures described herein.

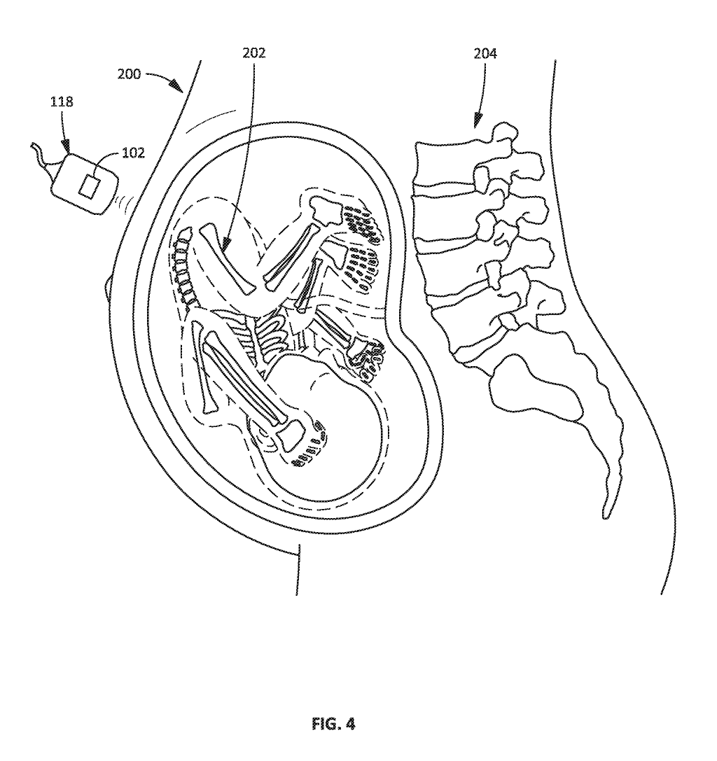

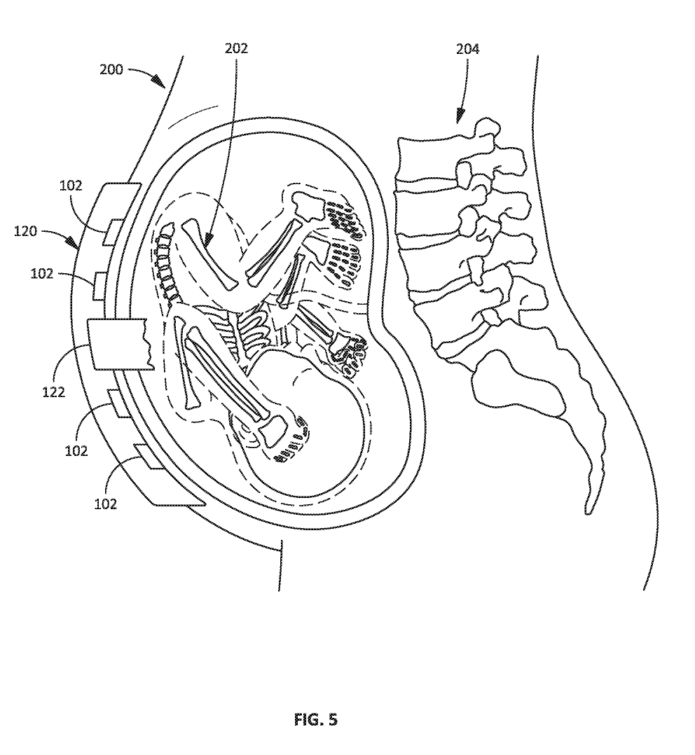

Referring to FIGS. 4 and 5, the one or more ultrasound elements 102 is configured to apply one or more ultrasonic signals to a body 200. The one or more ultrasound elements 102 can also be configured to receive one or more ultrasonic signals from the body 200. In some embodiments, an ultrasound element 102 is configured for both of applying one or more ultrasonic signals to the body 200 and receiving one or more ultrasonic signals from the body 200. In some embodiments, a first ultrasound element 102 can apply one or more ultrasonic signals to the body 200, and a second ultrasound element 102 can receive one or more ultrasonic signals from the body 200. In some embodiments, one or more ultrasound elements 102 from a first array 104 can apply one or more ultrasonic signals to the body 200, and one or more ultrasound elements 102 from a second array 104 can receive one or more ultrasonic signals from the body 200.

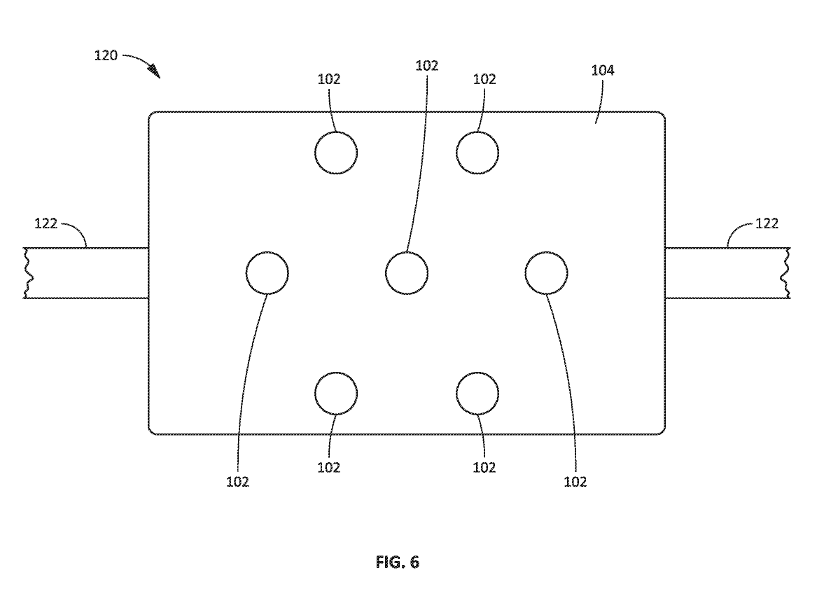

As shown in FIG. 4, the one or more ultrasound elements 102 can be included in an ultrasound scanner 118. For example, the one or more ultrasound elements 102 or arrays 104 can be included in a handheld ultrasound scanner, a scanning bed, or an ultrasound scanner supported by a robotic arm or by a fixed or adjustable support structure. As shown in FIGS. 5 and 6, the one or more ultrasound elements 102 or arrays 104 can be included in or coupled to a wearable garment 120. For example, the garment 120 can include one or more support members 122 (e.g., straps, belts, sleeves, or other garment-to-body connectors) that facilitate wearing of the garment 120 on the body 200. The one or more support members 122 can further facilitate appropriate positioning or contact between the one or more ultrasound elements 102 and the body 200. In some embodiments, the one or more support members 122 are adjustable to accommodate different body shapes and sizes or provide enhanced control of the position or placement of the one or more ultrasound elements 102 relative to the body 200. In some embodiments, the one or more ultrasound elements 102 are repositionable within the garment 122.

The one or more received ultrasonic signals can be processed to identify or image one or more portions 202 of a fetus within the body 200. In some embodiments, detected portions 202 of the fetus are identified as being portions of the fetus, and in some embodiments, the portions 202 are further identified as being or corresponding to particular features (e.g., bones, muscles, joints, etc.) of the fetus. For example, the one or more ultrasound elements 102 or arrays 104 can be configured for ultrasonography, shear wave elastography, or any other ultrasonic imaging technique. Examples of two-dimensional and three-dimensional ultrasonography are discussed in U.S. Pat. Nos. 8,352,059 and 8,105,240, U.S. Patent App. Pub. Nos. 2010/0082147 and 2007/0239006, and Michailidis, G. D., Papageorgiou, P., and Economides, D. L., “Assessment of fetal anatomy in the first trimester using two- and three-dimensional ultrasound,” The British Journal of Radiology, 75(891), 215-219 (2002). Examples of shear wave elastography are discussed in Evans et al., “Quantitative shear wave ultrasound elastography: initial experience in solid breast masses,” Breast Cancer Research, 12:R104 (2010), “Ultrasound shear wave imaging for bone” Ultrasound in Medicine & Biology, 26 (5), 833-837 (2000), and “Ultrasound shear wave imaging,” AIP Conf. Proc., 509, 847-852 (1999). Also, see VIRTUAL TOUCH quantification technology by Siemens Corporation. The foregoing patent and non-patent references are incorporated herein by reference. The one or more received ultrasonic signals can include one or more signal components of the applied ultrasonic signals. For example, the one or more received ultrasonic signals can include a reflected portion (e.g., an echo) of the one or more applied ultrasonic signals. The one or more received ultrasonic signals can also be responsive to the one or more applied ultrasonic signals. For example, the one or more received ultrasonic signals can include one or more secondary signals (e.g., shear waves) resulting from the application of the one or more applied ultrasonic signals to the body 200. In some embodiments, one or more shear waves transmitted through the one or more portions of the body 200 can include primary signals or portions thereof. For example, the one or more shear waves can include components of the applied ultrasonic signals. The one or more received ultrasonic signals can also include a reflected portion of the one or more applied ultrasonic signals that is associated with or affected by (e.g., resulting from an interaction with) one or more shear waves propagated through one or more portions of the body 200 as a result of the one or more applied ultrasonic signals. For example, the one or more received ultrasonic signals can include an echo with at least one parameter that is associated with a shear wave transmitted through at least a portion of the body 200 as a result of the one or more applied ultrasonic signals.

The controller 106 can be programmed or configured to receive one or more electrical signals or digital values associated with the one or more ultrasonic signals received by the one or more ultrasound elements 102. The one or more electrical signals or digital values can include at least one electrical signal or digital value associated with the one or more ultrasonic signals received by one ultrasound element 102. The one or more electrical signals or digital values can include at least one electrical signal or digital value associated with a combination of ultrasonic signals received by two or more ultrasound elements 102 of an array 104. The one or more electrical signals or digital values can include at least one electrical signal or digital value associated with a combination of ultrasonic signals received by two or more arrays 104. The controller 106 can be programmed or configured to perform ultrasonography, shear wave imaging, or mathematical analysis with the one or more received electrical signals or digital values to identify or image one or more portions 202 of the fetus.

In some embodiments, the controller is configured to identify one or more portions 202 of the fetus based upon a detection time (e.g., time of travel) associated with the one or more received ultrasonic signals. For example, the controller can filter out one or more echoes associated with non-fetal (e.g., maternal) portions 204 of the body 200 based upon the timing of the one or more received ultrasonic signals. Time gating can be used to ignore or discard a reflected portion of the one or more applied ultrasonic signals that are returned too early or too late to be associated with the fetus.

In some embodiments, the controller is configured to identify one or more portions 202 of the fetus based upon a comparison of a first signal portion and a second signal portion of the received one or more ultrasonic signals. For example, the first signal portion can be associated with one or more non-fetal portions 204 of the body, and the second signal portion can be associated with the one or more portions 202 of the fetus. The controller can compare at least one attribute of the first signal portion to at least one attribute of the second signal portion. For example, the controller can compare a frequency, amplitude, phase, velocity, or non-linear characteristic of the first signal portion to a frequency, amplitude, phase, velocity, or non-linear characteristic of the second signal portion. By way of further example, the controller can compare a reflectance value or signal scattering value of the first signal portion to a reflectance value or signal scattering value of the second signal portion. In some embodiments, one or more compared attributes can include or can be associated with elasticity values of the one or more portions 202 of the fetus and the one or more non-fetal portions 204 of the body 200. For example, the one or more received ultrasonic signals can include or can be associated with shear waves transmitted through the body 200 as a result of the applied ultrasonic signals.

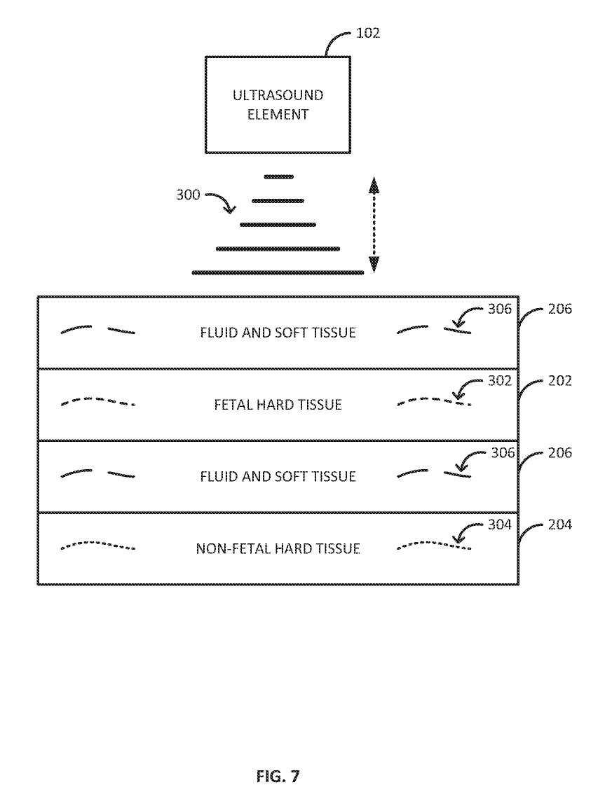

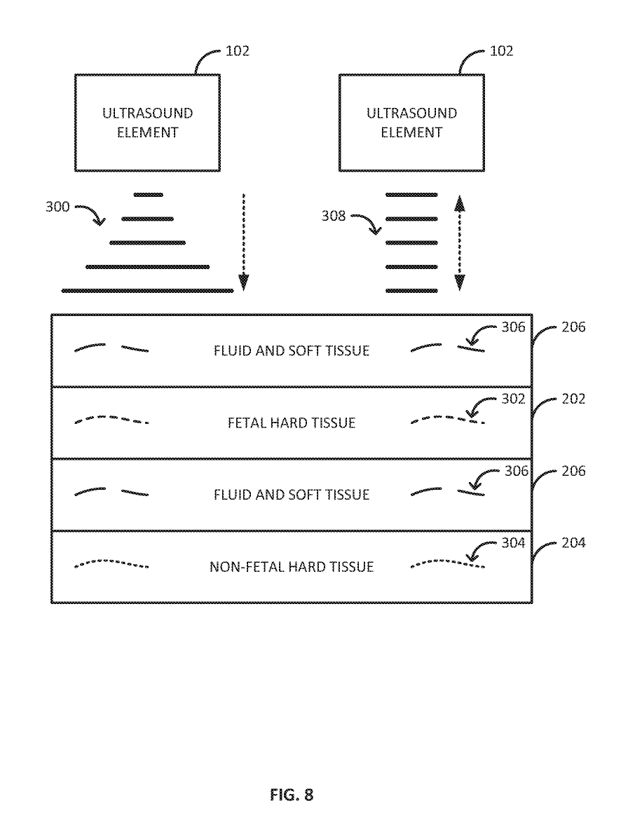

Embodiments of the fetal imaging device 100 configured for shear wave elastography are shown in FIGS. 7 and 8. Referring to FIG. 7, the one or more ultrasound elements 102 can apply one or more ultrasonic pulses 300 that cause one or more shear waves to be transmitted through the body 200. The one or more ultrasound elements 102 can detect shear waves resulting from the one or more applied ultrasonic signals. In addition to or instead of detecting the shear waves, the one or more ultrasound elements 102 can detect reflected portions (e.g., echoes) of the one or more applied ultrasonic signals. In some embodiments, one or more parameters of the reflected portions of the one or more applied ultrasonic signals are associated with the propagation of one or more shear waves through the one or more portions of the body 200. For example, one or more attributes of the one or more received ultrasonic signals can be indicative of a frequency, amplitude, phase, velocity, or non-linear characteristic of the one or more shear waves transmitted through the body 200. FIG. 8 shows an embodiment of the fetal imaging device 100 including one or more ultrasound elements 102 configured to apply one or more ultrasonic “push” pulses 300 that cause one or more shear waves to propagate through one or more portions of the body 200. The fetal imaging device 100 can also include one or more ultrasound elements 102 configured to apply one or more ultrasonic “detection” pulses 308. The one or more ultrasound elements 102 can receive one or more reflected portions of the one or more detection pulses 308. The one or more reflected portions can include one or more attributes associated with one or more attributes of the shear waves.

Different portions of the body 200 can be associated different elasticity values that affect the propagation of shear waves. For example, shear wave propagation can be affected by stiffness of the one or more portions of the body 200 through which the shear wave is propagating. The controller 106 can be programmed or configured to identify one or more portions 202 of the fetus based upon one or more attributes associated with the propagation of at least one shear wave 302 through the one or more portions 202 of the fetus. For example, one or more portions 202 of the fetus including hard tissue (e.g., fetal skeletal structures) can be distinguished from fluid or soft tissue 206 based upon a comparison of at least one attribute associated with one or more shear waves 302 transmitted through the one or more portions 202 of the fetus and at least one attribute associated with one or more shear waves 306 transmitted through the fluid or soft tissue 206. Because portions 202 including fetal hard tissue are stiffer than fluid and soft tissue 206, shear waves will propagate through the portions 202 differently (e.g., faster) than propagation through the fluid and soft tissue 206. By way of further example, one or more portions 202 of the fetus including hard tissue can be distinguished from one or more portions 204 of the body 200 including non-fetal hard tissue based upon a comparison of at least one attribute associated with one or more shear waves 302 transmitted through the one or more portions 202 of the fetus and at least one attribute associated with one or more shear waves 304 transmitted through the one or more portions 204 of the body 200 including the non-fetal hard tissue. Because portions 204 including non-fetal hard tissue are often stiffer than portions 202 including fetal hard tissue, shear waves may propagate through the non-fetal portions 204 differently (e.g., faster) than propagation through the portions 202 of the fetus.

The controller 106 can be programmed or configured to identify fetal skeletal tissue (e.g., bone tissue and/or cartilage) based on an elastography algorithm to differentiate maternal skeletal tissue from the fetal skeletal tissue. For example, the controller 106 can be programmed or configured to differentiate maternal skeletal tissue (e.g. bone tissue or cartilage of non-fetal portions 204) from fetal skeletal tissue (e.g., bone tissue or cartilage of fetal portions 202) based on a comparison of at least one attribute associated with one or more shear waves (e.g., shear waves 302 and 304) transmitted through the maternal skeletal tissue and the fetal skeletal tissue. Maternal bones or other maternal skeletal tissue (e.g., cartilage) can be stiffer than fetal bones and other fetal skeletal tissue; accordingly, shear waves 304 traveling through the maternal skeletal tissue can have different attributes (e.g., higher propagation speed/rate or differing attenuation rate) than shear waves 302 traveling through the fetal skeletal tissue. The controller can be programmed or configured to identify the fetal skeletal tissue and also identify or filter out the maternal skeletal tissue based on the comparison of one or more attributes (e.g., propagation speed/rate, attenuation rate, phase shift, and/or any other variable signal attribute) of the one or more shear waves (e.g., shear waves 302 and 304) propagated through portions 204 of the maternal body 200 and portions 202 of the fetus contained therein.

In some embodiments, the controller 106 can differentiate the fetal skeletal tissue from the maternal skeletal tissue based on shear wave elastography (e.g., as described above) or another ultrasonic imaging technique and can infer an identification of one or more portions 202 of the fetal skeleton based on a connectivity model for the fetus. For example, the controller 106 can be programmed or configured to identify the detected portions 202 of the fetal skeletal tissue by performing an image comparison between the detected (ultrasonically imaged) portions 202 of the fetus and one or more skeletal (e.g., bone tissue) connectivity models for the fetus. In some embodiments, the one or more connectivity models include a plurality of tissue connectivity models for a fetal skeleton or portions thereof in a plurality of different poses or at different viewing angles, and so forth. In some embodiments, the one or more connectivity models also include one or more tissue connectivity models for a maternal body or skeleton. For example, the one or more connectivity models can include tissue connectivity models for one or more portions of the fetal skeleton relative to one or more portions of the maternal skeleton. The controller 106 can be programmed or configured to infer an identification of the one or more portions of the fetus based on a comparison between detected portions of the fetus, detected portions of the mother, and one or more connectivity models for the fetus and the mother, where the posture of the maternal skeleton can inform the controller 106 of the likelihood of one or more potential postures of the fetus.

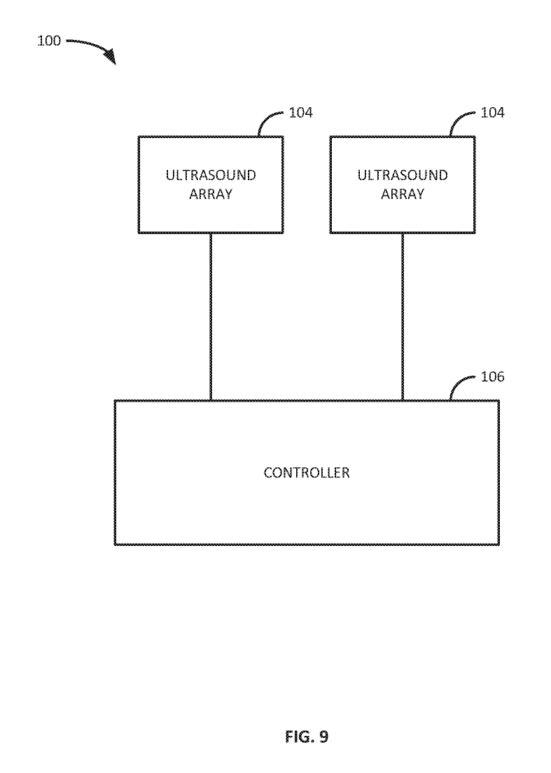

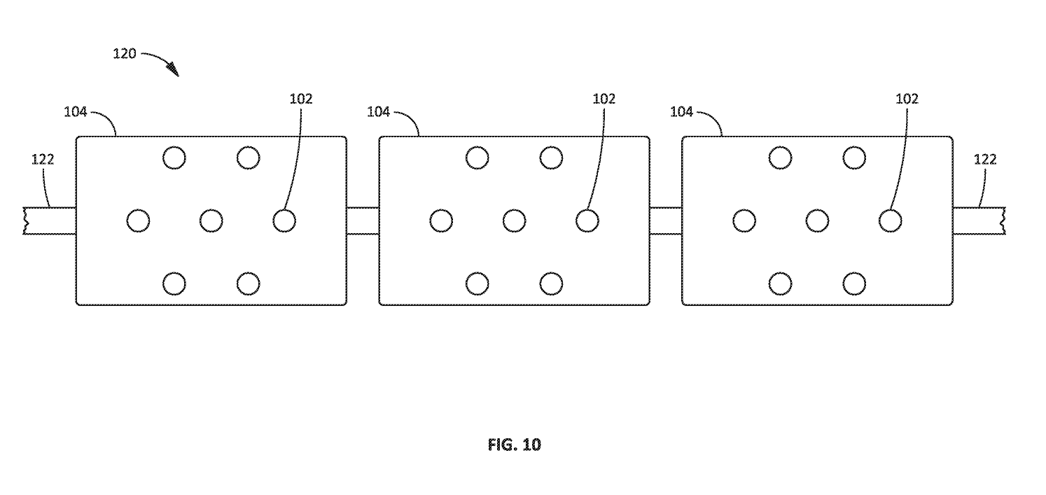

As shown in FIGS. 9-11, the fetal imaging device 100 can include a plurality of ultrasound arrays 104. In some embodiments, the fetal imaging device 100 includes independently addressable ultrasound arrays 104. For example, at least one array 104 can be operable independent of another one of the arrays 104. In some embodiments, two or more of the arrays 104 can also be spatially separated from one another. For example, FIG. 10 shows an embodiment of the wearable garment 120 including a plurality of arrays 104 that are spatially separated from one another. By way of further example, the fetal imaging device 100 can include two or more of: at least one array 104 positionable over a front of the body 200, at least one array 104 positionable over a first side of the body 200, at least one array 104 positionable over a second side of the body 200, and at least one array 104 positionable over a back of the body 200. In some embodiments, the fetal imaging device 100 can include two or more arrays 104 configured for tomographic imaging. For example, the controller 106 can ultrasonically image slices corresponding to one or more portions 202 of the fetus based upon ultrasonic signals received sequentially or concurrently by different arrays 104 or different portions of the arrays 104. The controller 106 can assemble the ultrasonically imaged slices to generate a combined three-dimensional image of the one or more portions 202 of the fetus. One or more ultrasonic signals can be applied by a first array 104 and received by the same array 104, a second array 104, or by both of the first and second arrays 104.

The controller 106 can be programmed or configured to monitor signals from ultrasound elements 102 of the one or more arrays 104 that indicate the quality of their acoustic coupling with the body 200. The controller 106 can apply a weighting to the electrical signals or data values received from the ultrasound elements 102 based upon the detected quality of acoustic coupling of one or more of the ultrasound elements 102. For example, methods of monitoring quality of acoustic coupling for transducers and weighting the transducers accordingly are discussed in U.S. Patent App. Pub Nos. 2014/0058263 and 2014/0058264, which are incorporated herein by reference.

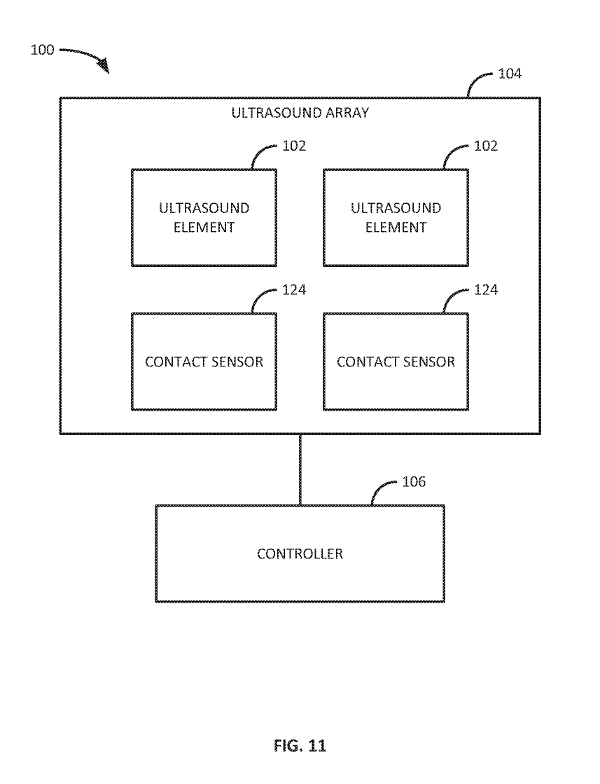

In some embodiments, the quality of acoustic coupling at each ultrasound element 102 is determined utilizing the ultrasound element 102, itself. For example, one or more ultrasonic signals applied or detected by the ultrasound element 102, either at the imaging frequency or at another frequency, can be used to determine the quality of acoustic coupling. As shown in FIG. 11, the fetal imaging device 100 can also include one or more contact sensors 124 associated with the one or more ultrasound elements 102. The one or more contact sensors 124 can include one or more contact force or pressure sensors, acoustic sensors, force measurement transducers, or capacitive or resistive sensors. In some embodiments, each ultrasound element 102 can be associated with a respective contact sensor 124. In some embodiments, an array 104 or group of elements 102 within the array 104 are associated with a respective contact sensor 124.

The controller 106 can be programmed or configured to monitor acoustic coupling of the one or more ultrasound elements 102 with the body 200, and can simply omit or discard imaging data received from an ultrasound element 102 that does not meet a contact quality threshold. In some embodiments, there can be a more complicated relationship between contact quality and imaging data. For example, data can be “downgraded” and used only if there is no data available from a nearby ultrasound element 102 in better contact with the body 200. In some embodiments, only the best 90% (or 70% or 50% or 30% or the like) elements 102 (i.e., the 90% of ultrasound elements 102 having the best acoustic contact with the body 200) are used to image the one or more portions 202 of the fetus. An image can also include false color to identify more or less “reliable” areas. For example, all measured image data can be used to generate an ultrasound image, but pixels can be shaded in red in areas where contact with the body 200 is poor, and in green in areas where it is good. In some cases, an ultrasonic image may have “holes” indicating that there was insufficient contact with the body in those areas, or the controller 106 can simply provide a signal indicating that no image can be produced at all because of poor contact quality.

Monitoring of contact with the body 200 can be static or dynamic in nature. For a single image of a non-moving body, it may be sufficient to determine quality of acoustic contact once for the ultrasound elements 102 of an array 104. For a longer imaging process or a body in motion, it can be preferable to dynamically monitor the contact with various ultrasound elements 102 and to continuously adjust the resultant image or collected data, either by weighting the data and using “better” data more heavily, or by applying false color or similar cues to the image to indicate areas of better or worse image quality. Data weighting can be computational in nature (where, for example, pixels “count” more heavily when they are considered to be more reliable), or it can be accomplished by providing more power to one or more ultrasound elements 102 that appear to have a better quality of contact with the underlying body 200.

In some embodiments, once the quality of acoustic coupling is measured, the controller 106 can provide one or more indications or control signals in an attempt to remediate ultrasound elements 102 having poor acoustic coupling. For example, the controller 106 can provide an indication for a user or a control signal to a dispenser to dispense additional ultrasound gel (or another acoustic coupling agent) to try to improve the quality of coupling at an ultrasound element 102 having a poor acoustic coupling quality. By way of further example, the controller 106 can provide an indication for a user or a control signal to an actuator to adjust a contact force in the area having a poor contact rating.

Determining a quality of acoustic coupling at (or in the immediate proximity of) each ultrasound element 102 can include, for example, determining the magnitude of an echo or reflection from an exterior surface of the body 200, either at the imaging frequency or at a different frequency. A strong echo is typically associated with good coupling with the body 200. Determining a quality of acoustic coupling can include measuring contact with the body 200 by measuring force, resistance, capacitance, or some other property. For example, a strain gage can be placed at the contact surface of an ultrasound element 102 to measure the force between that element 102 and the body 200, where a larger contact force is typically indicative of a better quality of acoustic coupling.

Once acoustic coupling data has been determined for an array 104, the data can be used to apply a weighting to the ultrasound elements 102 making up the array 104. For example, the measured qualities (e.g., contact forces) can be sorted, and the bottom 30% can be discarded. Another threshold can equally well be substituted, either as a different percentage or as a different absolute value of the quality measure. In some embodiments, explicitly discarding measurements from sites in poor contact will improve image reconstruction compared to the alternative of receiving a low or null signal due to poor coupling quality and assuming that this value represents an actual trough in the arriving ultrasonic wave. The remaining sensor data can be used to generate an ultrasound image. In some embodiments, the ultrasound elements 102 associated with the discarded signals can create “holes” in the ultrasound image. The data can be interpolated from neighboring ultrasound elements 102 to show estimate the missing portions. In embodiments where the data is interpolated to produce a complete image, false color can be used to identify interpolated regions.

In some embodiments, the quality of acoustic coupling at an ultrasound element 102 can be used to determine a “spot size” for data from that element 102. Ultrasound elements 102 with relatively good coupling can have large spots, while those with relatively poor coupling have smaller spots. In some embodiments, spot sizes can go to or near zero for sufficiently poor acoustic coupling. When displaying an image, any given pixel is displayed using the ultrasonic measurement generated by the ultrasound element 102 having that pixel in its “spot.” If a pixel appears in multiple spots, the controller 106 can either use an average of the values of the spots overlapping the pixel, or it can use the largest spot that overlaps it. Pixels falling outside the spots can have no data associated with them.

In some embodiments, determination of the acoustic coupling quality can be used to adjust one or more power levels for the ultrasound elements 102. This can include, for example, increasing power to sources having good contact, or decreasing (or turning off entirely) sources having poor contact. This can include increasing power to sources having poor contact so as to maintain or equalize a desired level of acoustic coupling with the body 200. In some embodiments, the determined quality of acoustic coupling for an ultrasound source can be used to determine a measure of how much of its output actually couples into the body; this can then be used within data analysis algorithms by replacing source distributions based on emitted power by ones based on coupled power.

In some embodiments, knowledge of the spatial profile of coupling quality for an array of ultrasound elements 102 can be used in operation of a phased array. The activation of individual sources can be based upon the spatial pattern of those having sufficient coupling quality, and the power delivered to each source can depend upon its coupling quality in order to achieve a desired spatial pattern of body-coupled ultrasonic power. Similarly, the spatial pattern of the coupling quality can also be used in determining the reception properties of a phased array. Array elements 102 having poor coupling quality can be deleted from the antenna pattern, and received ultrasonic signals at a location can be divided by the coupling quality to provide a better measure of the ultrasonic signal arriving at the surface of the body 200.

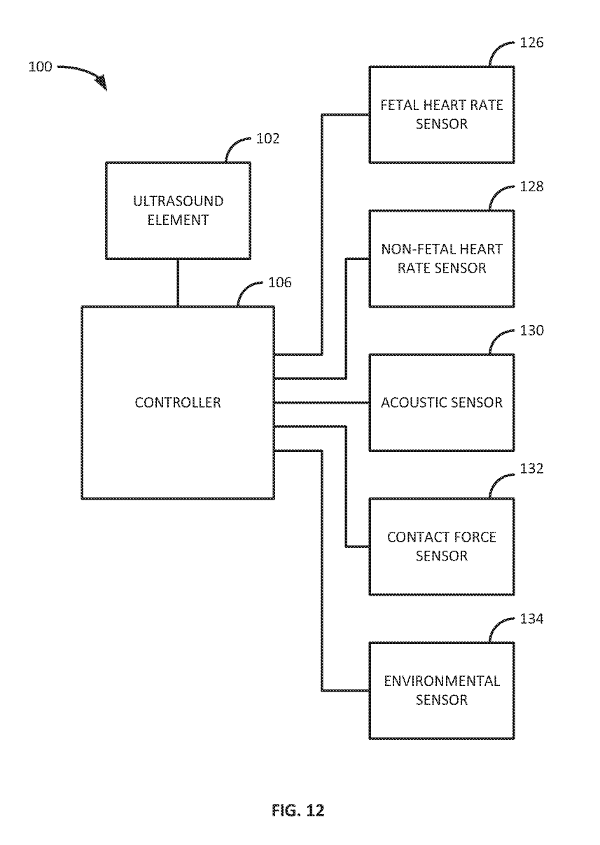

An embodiment of the fetal imaging device 100 is shown in FIG. 12 to include one or more sensors in addition to the one more ultrasound elements 102. In some embodiments, the fetal imaging device 100 includes one or more physiology sensors. The one or more physiological sensors can provide information about the fetus or the body 200 bearing the fetus through contact with the skin or proximity to the skin of the body 200. A physiological sensor can include at least one of a heart rate sensor, a respiratory sensor, a thermal sensor, a blood pressure sensor, a hydration sensor, an oximetry sensor, an electrocardiograph, an electroencephalograph, or an electromyograph. For example, the one or more physiology sensors can include a fetal heart rate sensor 126 configured to detect a heart rate of the fetus or a non-fetal heart rate sensor 128 configured to detect a non-fetal heart rate associated with the body 200.

In some embodiments, the fetal imaging device 100 includes one or more fetal activity sensors, such as an acoustic sensor 130, accelerometer, contact force sensor 132, or other force, motion, proximity, or pressure sensor configured to detect at least one of a vibration, applied force, or motion indicative of fetal activity. For example, detected vibrations or pulses at a surface of the body 200 can be associated with movement (e.g., kicking or repositioning) of the fetus within the body 200. In addition to or instead of the one or more fetal activity sensors, the controller 106 can track fetal activity based upon imaging data collected via the one or more ultrasound elements 102. For example, the controller 106 can detect fetal activity by identifying a difference between a first image of the one or more portions 202 the fetus and a second image of the one or more portions 202 of the fetus. The difference between the first image and the second image can be associated with a movement of the fetus. In some embodiments, the fetal activity can also be based on information communicated to the controller 106 from the physiology sensors. For example, the controller 106 can be programmed or configured to assess a level of fetal activity based on one or more fetal physiological parameters including one or more of: a fetal heartrate, in utero pressure, in utero position of the fetus, in utero temperature, average movement of the fetus, or the like. In some embodiments, the controller 106 can detect fetal activity by identifying a posture of the fetus based upon one or more identified portions of the fetus. For example, the one or more identified portions of the fetus can match up with an anatomical alignment or connectivity model stored in a database accessible by the controller 106. In some embodiments, the controller can identify a posture of the fetus based upon an ultrasound image of the one or more portions of the fetus. For example, the controller 106 can perform image recognition analysis to map the ultrasound image collected via the one or more ultrasound elements 102 to one or more stored images that are associated with identified fetal postures. The stored images can be computer-generated images, images of the fetus, or images of a model or another fetus.

In some embodiments, the fetal imaging device 100 includes one or more environmental sensors 134 configured to detect one or more environmental attributes external to the body. For example, the one or more environmental sensors 134 can include an illumination sensor (e.g., photoresistor, photodiode, or camera), an acoustic sensor (e.g., transducer or microphone), a timer, a location device (e.g., GPS), a temperature sensor (e.g., thermistor or thermometer), a pressure sensor, an altimeter, a moisture sensor, or the like. Examples of the one or more environmental attributes can include illumination intensity, illumination spectrum, sound amplitude, sound spectrum, temperature, pressure, altitude, humidity level, location, time, date, image recognition attributes, voice recognition attributes, or other contextual information regarding the environment surrounding the body 200.

The controller 106 can be programmed or configured to receive data from the one or more sensors (e.g., sensor 126, sensor 128, sensor 130, sensor 132, or sensor 134). For example the controller can receive one or more electrical signals or digital values associated with physiology data, fetal activity data, or environmental data from the one or more sensors. In some embodiments, this data can be received by the controller 106 through a multiplexer. The controller 106 can store sensor data to a local or remote memory. The controller 106 can transmit the sensor data to a second device or a remote memory. In some embodiments, the controller 106 is also configured to send control signals to the one or more sensors to active or deactivate the sensors, to adjust one or more sensor parameters, or to request sensor data. For example, the controller 106 can calibrate a sensor by sending a control signal to the sensor. The controller 106 can also turn a sensor on or off. In some embodiments, the controller can generate a mapping between at least two imaging or sensor data parameters. For example, the controller can generate a mapping between at least two of: an image of the one or more portions of the fetus, an identified posture of the fetus, a level of fetal activity, an environmental attribute external to the body, a heart rate of the fetus, or a heart rate associated with the body. The mapping can be a time-indexed mapping or a one-to-one mapping of concurrently or sequentially collected data.

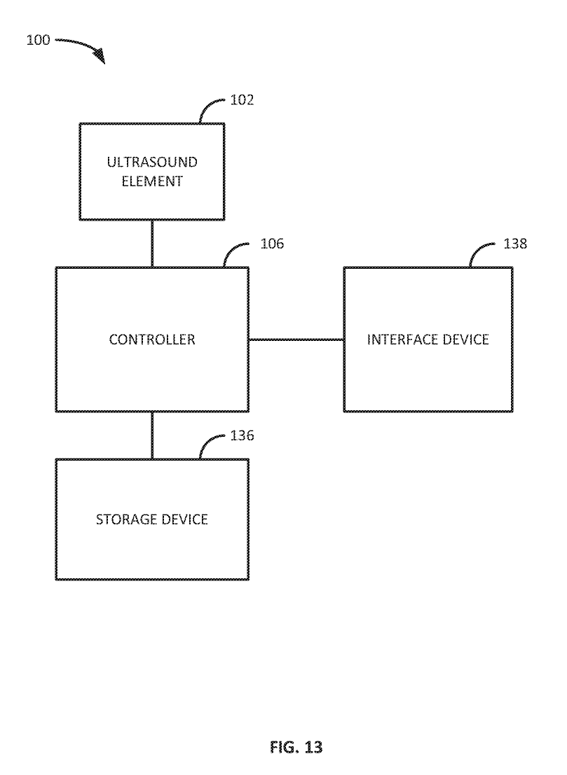

As shown in FIG. 13, the fetal imaging device 100 can further include a storage device 136 configured to store data associated with the one or more received ultrasonic signals (e.g., ultrasound imaging data) or other sensor data (e.g., data from sensor 126, sensor 128, sensor 130, sensor 132, or sensor 134). The storage device can include random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other non-transitory medium which can be used to store the imaging or sensor data collected by the controller 106 via the one or more ultrasound elements 102 or one or more additional sensors.

The controller 106 can be programmed or configured to initiate the generation of one or more images of the one or more portions 202 of the fetus by commanding at least one ultrasound source to apply one or more ultrasonic signals to the body. The controller 106 can command that the ultrasonic signals be applied periodically, based upon a schedule, or in response to at least one of a user input, a request received from a second device, or an occurrence of a predetermined event (e.g., detecting fetal activity above or below a threshold fetal activity).

The controller 106 can be programmed or configured to record one or more images of the one or more portions 202 of the fetus to the storage device 136. The images can be based upon the one or more received ultrasonic signals. For example, the recorded images can include recorded ultrasound echoes or computer-generated images that are based upon the electrical signals or data values associated with the one or more received ultrasonic signals. The controller 106 can record the one or more images of the one or more portions 202 of the fetus periodically, based upon a schedule, or in response to at least one of a user input, a request received from a second device, or an occurrence of a predetermined event (e.g., detecting fetal activity above or below a threshold fetal activity). The controller 106 can also be configured to record a second image based upon a first recorded image. For example, the controller 106 can record a second image of one or more portions 202 of the fetus when the first recorded image is associated with an identified posture of the fetus or viewing angle of the fetus. The second image can be directed to obtaining another image with the same or similar posture or viewing angle as the first image, or the second image can be directed to obtaining an image with a different posture or viewing angle. By way of further example, the controller 106 can record the second image of the one or more portions of the fetus when the first recorded image is associated with a non-identifiable posture of the fetus or when the first recorded image includes a non-identifiable portion of the fetus. That is, the second image can include an image wherein the fetal posture or obscured/non-detected portion of the fetus can be identified.

In some embodiments, the controller 106 is configured to assign one or more posture or viewing angle identifications to the one or more recorded images of the one or more portions 202 of the fetus. For example, the controller can identify at least one of a posture (e.g., kicking, sleeping, curled up, stretched out) or a viewing angle (e.g., front, back, left, right) associated with a recorded image of the fetus based upon one or more identified portions of the fetus or a comparison between the recorded image and a computer-generated image, a previously stored image of the fetus, or a stored image of a model or another fetus.

In some embodiments, the controller 106 can be further configured to assign one or more rankings to the one or more recorded images of the one or more portions 202 of the fetus. For example, the one or more images can be ranked according to an image quality (e.g., higher/lower resolution), an identified posture, an ability to identify certain features (e.g., face, arms, legs, torso, or genitals) of the fetus, or according to a user input (e.g., a user selected ranking or “like” associated with the image). In some embodiments, the controller 106 can record a second image of one or more portions 202 of the fetus based upon an assigned ranking of a first recorded image of the fetus. For example, the controller 106 can record the second image based upon a user-driven score or ranking of the first image.

In some embodiments, the controller 106 can be further configured to discard at least one image of the one or more recorded images to save storage space or to filter out redundant or poor quality images. For example, the controller 106 can discard at least one image of the one or more recorded images periodically, based upon a schedule, or in response to at least one of a user input, a request received from a second device, or an occurrence of a predetermined event (e.g., detecting fetal activity above or below a threshold fetal activity). The one or more discarded images can include low ranking images or those identified as having at least one of a redundant viewing angle, a redundant fetal posture, an obscured portion of the fetus, or an expired timestamp.

The fetal imaging device 100 can also include or can be coupled to an interface device 138 configured to display or otherwise communicate imaging data (e.g., ultrasound or computer-generated images of the fetus) or sensor data to a user. In some embodiments, the interface device 138 can also receive user inputs. User inputs can be received in the form of at least one of a physical input (e.g., pressing a button or entering a command), visually (e.g., via image recognition, motion recognition, or posture recognition), or audibly (e.g., voice commands). The interface device 138 can include a graphical user interface (GUI), a touchscreen assembly (e.g., a capacitive touch screen), a liquid crystal display (LCD), a light-emitting diode (LED) display, a projection-based display, an audio device, or the like. In some embodiments, the interface device 138 includes a mobile computing device (e.g., hand-held portable computer, Personal Digital Assistant (PDA), laptop computer, netbook computer, or tablet computer), mobile telephone device (e.g., cellular telephone or smartphone), a device that includes functionalities associated with a smartphone and a tablet computer (e.g., phablet), portable game device, portable media player, multimedia device, satellite navigation device (e.g., Global Positioning System (GPS) navigation device), e-book reader device (eReaders), Smart Television (TV) device, surface computing device (e.g., table top computer), or Personal Computer (PC) device. In some embodiments, the interface device 138 includes a display or audio device coupled to the controller 106 via one or more wired or wireless communication. For example, the interface device 138 can send or receive electrical communication signals (e.g., digital or analog signals), acoustic communication signals, optical communication signals, radio communication signals, microwave communication signals, infrared communication signals, ultrasonic communication signals, or the like.

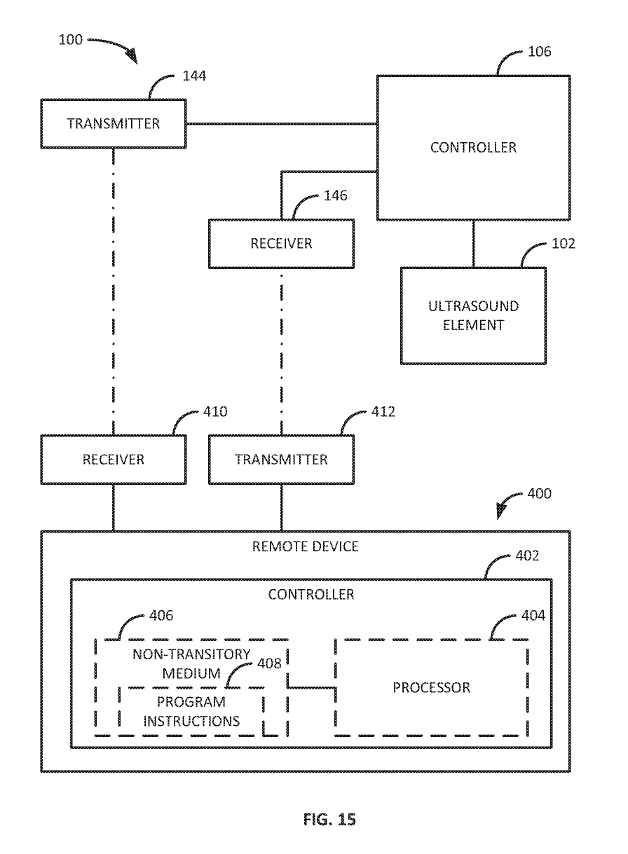

As shown in FIG. 15, the controller 106 can be coupled to a transmitter 144 for communicating with a remote device 400. For example, the transmitter 144 can send communication signals to a receiver 410 of the remote device 400. The transmitter 144 can include a wired or wireless transmitter. For example, the transmitter can transmit imaging data, sensor data, requests, control signals, pairing data, or the like via one or more of an electrical signal, a radio signal, a microwave signal, a terahertz signal, an infrared signal, an optical signal, an ultraviolet signal, a subsonic signal, an audible signal, an ultrasonic signal, or a magnetic signal. In some embodiments, the transmitter 144 can connect to the remote device 400 via a wireless network (e.g., WiFi, Zigbee, Bluetooth, etc.). In some embodiments, the transmitter 144 can be coupled with a connector for connecting to the remote device 400. Examples of a connector include a serial port, a serial cable, an IEEE 1394 interface, a parallel port, a parallel cable, a network (e.g., Ethernet) port, a network (e.g., Ethernet) cable, a Universal Serial Bus (USB) port, a USB cable, a fiber optic port, a fiber optic cable, or the like.

The controller 106 can also be coupled to a receiver 146 for communicating with the remote device 400. For example, the receiver 146 can receive communication signals from a transmitter 412 of the remote device 400. The receiver 146 can include a wired or wireless receiver. For example, the receiver 146 can receive database information, requests, control signals, pairing data, or the like from the remote device 400 via one or more of an electrical signal, a radio signal, a microwave signal, a terahertz signal, an infrared signal, an optical signal, an ultraviolet signal, a subsonic signal, an audible signal, an ultrasonic signal, or a magnetic signal. In some embodiments, the receiver 146 can connect to the remote device 400 via a wireless network (e.g., WiFi, ZigBee, Bluetooth, etc.). In some embodiments, the receiver 146 can be coupled with a connector for connecting to the remote device 400. Examples of a connector include a serial port, a serial cable, an IEEE 1394 interface, a parallel port, a parallel cable, a network (e.g., Ethernet) port, a network (e.g., Ethernet) cable, a Universal Serial Bus (USB) port, a USB cable, a fiber optic port, a fiber optic cable, or the like. In some embodiments, the transmitter 144 and the receiver 146 can be implemented in a single communications device or can share one or more components (e.g., a transmitting and receiving antenna).

The controller 106 can be programmed or configured to transmit imaging data (e.g., electrical signals, digital values, or images) based upon the one or more received ultrasonic signals to the remote device 400. For example, the transmitter 144 can transmit one or more images of one or more portions 202 of the fetus or sensor data to the remote device 400 periodically, based upon a schedule, or in response to at least one of a user input, a request received from a second device, or an occurrence of a predetermined event (e.g., detecting fetal activity above or below a threshold fetal activity). In some embodiments, the remote device 400 can be located at a medical facility or animal breeding facility or can include a fetal monitoring application (e.g., PC or smartphone application) configured to track fetal activity or development, or collect images for real-time, on-demand, or scheduled viewing. For example, the example the transmitter 144 can stream, in real-time, collected or computer-generated images of the one or more portions 202 of the fetus.

The remote device 400 can include a mobile computing device (e.g., hand-held portable computer, Personal Digital Assistant (PDA), laptop computer, netbook computer, or tablet computer), mobile telephone device (e.g., cellular telephone or smartphone), a device that includes functionalities associated with a smartphone and a tablet computer (e.g., phablet), portable game device, portable media player, multimedia device, satellite navigation device (e.g., Global Positioning System (GPS) navigation device), e-book reader device (eReaders), Smart Television (TV) device, surface computing device (e.g., table top computer), Personal Computer (PC) device, server, or cloud computing network. The remote device 400 can include a respective controller 402. The controller 402 can include a processor 404, such as a general-purpose processor, an application specific integrated circuit (ASIC), one or more field programmable gate arrays (FPGAs), a digital-signal-processor (DSP), a group of processors or processing cores, or other suitable electronic processing components. The processor 404 can be communicatively coupled to at least one non-transitory medium 406 (e.g., RAM, ROM, Flash Memory, hard disk storage, or the like) for storing data and program instructions 408 that enable the controller 402 to perform various operations described herein when executed by the processor 404. The non-transitory medium 406 can include non-transient volatile memory or non-volatile memory. The non-transitory medium 406 can include database components, object code components, script components, or any other type of information structure for supporting the various activities and information structures described herein. In some embodiments, the remote device 400, via controller 402, can perform one or more operations of controller 106 described herein. Controllers 106 and 402 can be interchangeable for various operations described herein. For example, controller 106 can be programmed to collect electrical signals or digital values from the one or more ultrasound elements 102, while an image based upon the signals or digital values is generated by controller 402. Other tasks can be divided between or accomplished jointly by the controller 106 of the fetal imaging device 100 and the controller 402 of the remote device 400 or by a controller of a second local device (e.g., local PC or server).

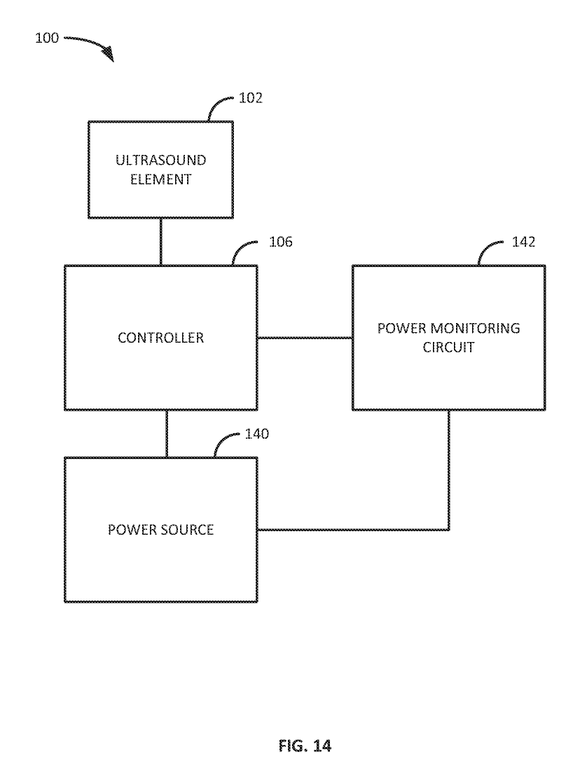

As shown in FIG. 14, a power source 140 can provide electrical power to the controller 106, the one or more ultrasound elements 102, and any other components of the fetal imaging device 100. In one embodiment, the power source 140 is a battery. For example, the power source 140 can be at least one of a disposable battery, rechargeable battery, or removable battery. In some embodiments, the power source 140 can allow recharging of power source 140 without decoupling the power source 140 from the fetal imaging device 100. For example, the power source 140 can be a rechargeable battery configured to be recharged through wireless changing (e.g., inductive or optical charging). In other embodiments, the power source 140 can receive direct or alternating current from a source outside the fetal imaging device 100. For example, the power source 140 can include an adapter or a transformer. In some embodiments, the power source 140 can receive power from a wireless source. For example, the power source 140 can include a coil configured to receive power through induction. Other examples of the power source 140 can include a capacitor that can be charged by a wired or wireless source, one or more photoelectric cells (e.g., solar cells), a metamaterial configured to provide power via microwaves, or the like.

In some embodiments, the controller 106 is coupled to a power monitoring circuit 142 that is configured to detect a battery life, power level, or other indication of available energy resources from the power source 140. The controller 106 can be programmed or configured to collect or record the imaging data or sensor data based on the detected battery life, power level, or other indication of available energy resources. For example, the controller 106 can be programmed or configured to collect a reduced number of images or collect images at a lower resolution when the detected battery life is below a threshold battery life. The controller 106 can also be programmed or configured to avoid discarding an image when the detected battery life is below a threshold battery life. For example, the controller 106 can be programmed or configured to store or continue to store an image that would otherwise be discarded instead of attempting to collect a better quality image or one associated with a preferred view or posture. The controller 106 can also be programmed or configured to transmit imaging data or sensor data via transmitter 144 to the remote device 400 based on the detected battery life, power level, or other indication of available energy resources. For example, the transmitter 144 can transmit a reduced number of images or transmit at a lower resolution or number of frames per second when the detected battery life is below a threshold battery life.

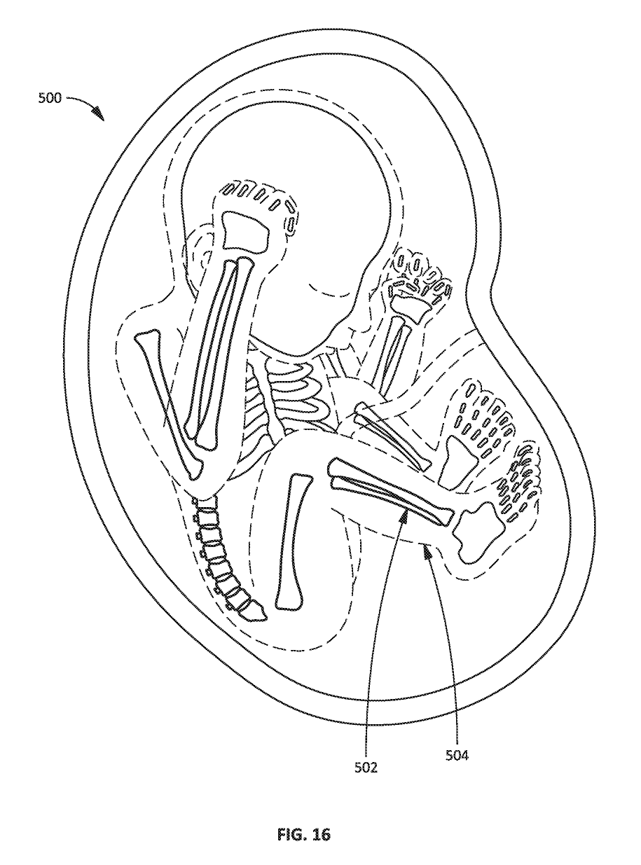

FIGS. 16-20 show images 500 based upon the one or more received ultrasonic signals. In some embodiments, the controller 106 can be programmed or configured to generate a two-dimensional or three-dimensional image of one or more portions 502 of the fetus. In some embodiments, a three-dimensional image can be rotatable with at least two degrees of freedom to provide multiple viewing angles. The controller 106 can be programmed or configured to convert electrical signals or data values received from the one or more ultrasound elements 102 into image pixels. The controller 106 can also be programmed or configured to identify one or more portions 502 of the fetus based upon the images or based on an analysis of the data. For example, the controller 106 can be programmed or configured to image or identify the one or more portions 502 of the fetus based upon shear wave elastography or time gating. The controller 106 can be programmed or configured to create a computer-generated image of the one or more identified or non-identified portions 502 of the fetus by applying one or more image processing filters (e.g., isolation, interpolation, or extrapolation filters) or based on stored anatomical models. For example, non-adjacent images of a portion of the fetus can be obtained and the non-imaged portion of the fetus adjacent to the imaged portions can be created by the controller through interpolation, extrapolation, etc. Moreover, the controller 106 can be programmed or configured to generate an image 500 of identified portions 502 (e.g., hard tissue portions) of the fetus with non-fetal (e.g., maternal) structures removed from the image 500.

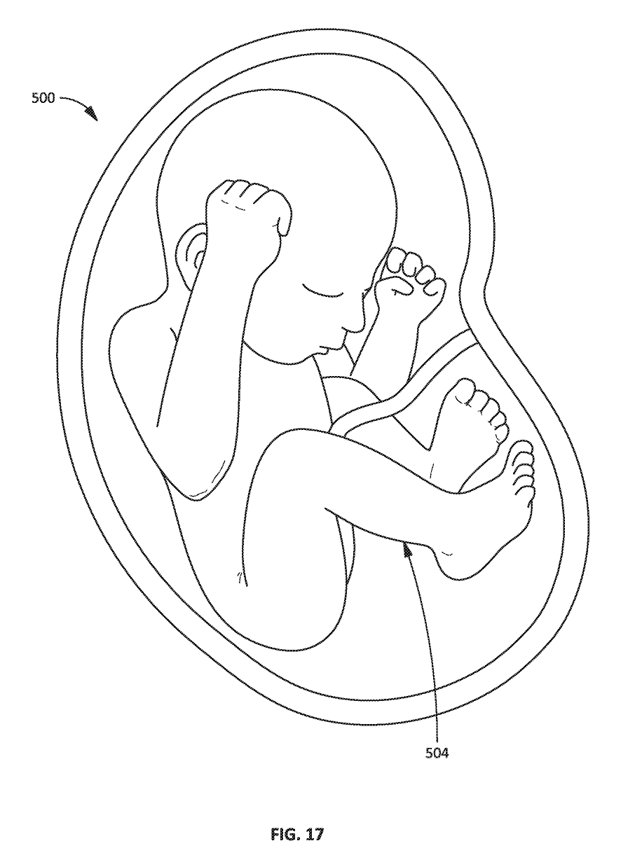

As shown in FIG. 17, the controller 106 can be programmed or configured to generate a textured image 500 with flesh mapped onto the one or more identified portions 502 of the fetus. For example, the controller 106 can apply a texture map 504 to the identified portions 502 to generate the textured image 500 of the fetus. In some embodiments, the texture map 504 includes a predetermined texture map. In some embodiments, the texture map 504 is based on a specified or detected genomic element, skin tone, demographic, gender, or development stage associated with the fetus. In some embodiments, the genomic element can be that of the fetus, or that of a relative of the fetus (e.g., a parent, grandparent, sibling, etc.). The genomic element can be a portion of a gene, a single gene, a plurality of genes, a haplotype, an allele, one or more non-coding DNA sequences, or an epigenetic element, and can represent nuclear or mitochondrial genomic elements. For example, the genomic element can be one previously correlated with appearance, with musculature, with fat content, with fetal health, or the like. In some embodiments, the texture map is at least partially based on an image of a person. For example, the texture map can be based upon an image of at least one relative, such as a parent, sibling, uncle, aunt, cousin, grandparent, or the like. The controller 106 can map a facial expression onto the image 500 of the fetus. For example, the controller 106 can map a randomly selected or randomly generated facial expression onto the image 500 of the fetus. In some embodiments, the facial expression can be based upon a time (e.g., day or night), date, location (e.g., home, park, hospital, etc.), level of fetal activity, identified fetal posture, or environmental attribute (e.g., detected sound, motion, temperature, or weather condition). In some embodiments, the facial expression can be associated with a user input. For example, the facial expression can change according to a data input or selection or in response to a detected audio or visual input associated with the user, such as an identified voice, musical rhythm, facial feature, gesture, phrase, or other perceptible characteristic or action of the user. By way of further example, a satisfied facial expression (e.g., a smile) can be mapped onto the image 500 of the fetus when a mother's face is detected or voice is heard. Viewing a facial expression can allow a viewer to feel as though she is interacting with the fetus, thereby providing her with reassurance or other positive emotions. In this regard, the fetal imaging device 100 can be used to treat symptoms that can affect expecting mothers, such as anxiety, paranoia, or depression.

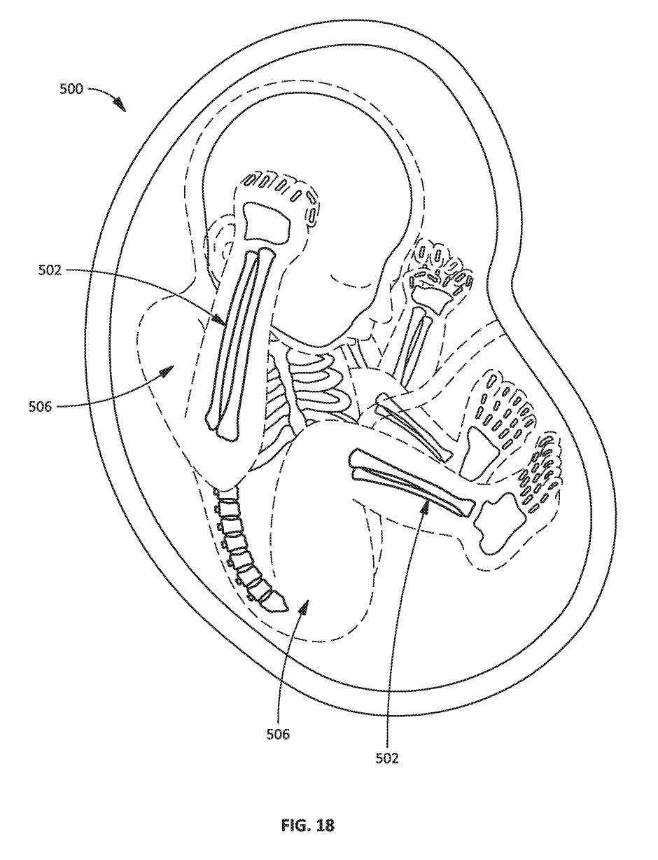

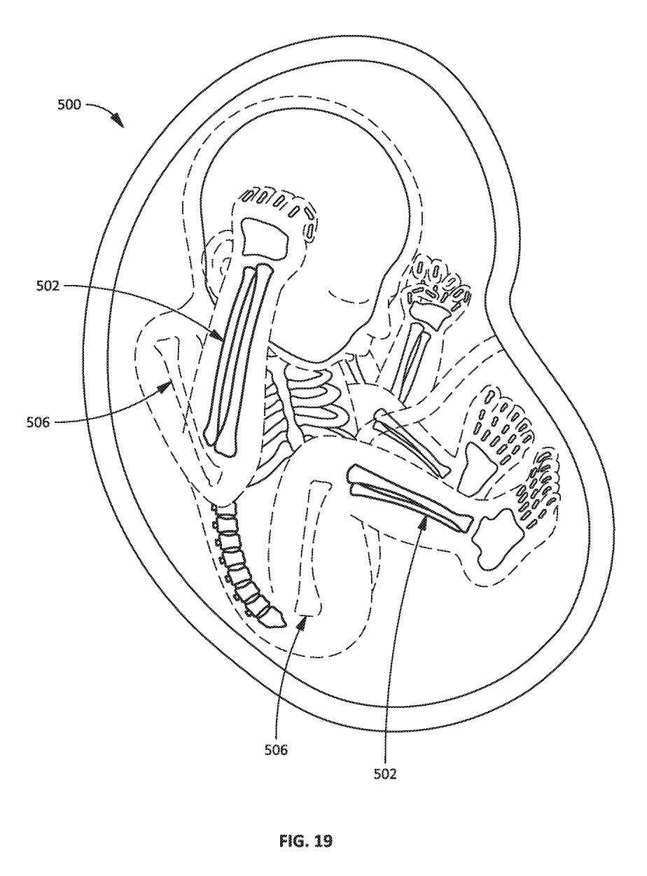

As shown in FIGS. 18 and 19, one or more portions 506 of the fetus may be non-detectable. In some embodiments, controller 106 is programmed or configured to determine when at least one portion 506 is not detectable by the one or more ultrasound elements 102 or when the at least one portion 506 can be only partially detected, is partially or totally obscured, or can only be detected at a low imaging quality, for example, such that it cannot be identified without referencing one or more identified portions 502 of the fetus. The controller 106 can be programmed or configured to provide an instruction for imaging the non-detected portion 506 of the fetus from an alternative imaging angle in an attempt to receive any imaging data or higher quality imaging data for the non-detected portion 506. In some embodiments, the controller 106 can be programmed or configured to image the non-detected portion 506 of the fetus from an alternative imaging angle utilizing a different ultrasound element 102, array 104, or combination of ultrasound elements 102 from one or more arrays 104.

In some embodiments, the controller 106 can be programmed or configured to infer an identification of one or more non-detected portions 506 based on an identification of one or more detected portions 502 of the fetus. For example, the controller 106 can be programmed or configured to infer an identity or location of the one or more non-detected portions 506 of the fetus based upon the identities, locations, and/or orientations of the one or more detected portions 502 of the fetus. The one or more non-detected portions 506 of the fetus can include one or more non-detected portions of an anatomical structure (e.g., bone or muscle) that includes the one or more detected portions 502 of the fetus, or the one or more non-detected portions 506 can include one or more portions of a different anatomical structure (e.g., a second bone or muscle) from the one or more detected portions 502 (e.g., one or more portions of a first bone or muscle) of the fetus.

The controller 106 can be programmed or configured to compute a depth map based on the one or more ultrasonic signals received from body. In some embodiments, the one or more ultrasound elements 102 can apply a structured or patterned ultrasonic signal or cluster of signals. For example, applied signals can include signals that define a pattern based on one or more signal attributes, such as signal strength, frequency, phase, timing (e.g., timing of pulsed signals), and so forth. Portions of the ultrasonic signal or signals that are reflected, scattered or transmitted by the fetus can then be detected by the one or more ultrasound elements 102. The controller can be programmed or configured to compute the depth of various portions of the fetus compared to one another based on deformations in the detected ultrasonic pattern as compared with the applied ultrasonic pattern. In some embodiments, the controller 106 can compute the relative depths based on time of flight, signal attenuation, or other factors associated with the detected ultrasonic signals.

The controller 106 can be programmed or configured to identify one or more portions 502 of the fetus (e.g., determine that a detected portion of the fetus corresponds to a particular feature or a group of possible features) based on received ultrasonic signals. For example, the controller 106 can determine bones or muscles corresponding to the detected portions 502 of the fetus. The controller 106 can also be programmed or configured to infer an identification of one or more non-detected portions 506 of the fetus based on the identification of the detected portions of the fetus and a computed depth map that is based on the arrangement of the detected portions 502 of the fetus. For example, the depth map can be compared with a library of possible skeletal arrangements for the fetus. In some embodiments, the controller 106 is programmed or configured to infer the skeletal arrangement (e.g., pose or posture) of the fetus, and can therefore infer the one or more non-detected portions of 506 of the fetus, with one or more decision trees or a randomized decision forest (e.g., including two or more decision trees selected from a group of decision trees). For example, the controller 106 can perform a series of determinations to identify anatomical structures (e.g., bone, cartilage, or muscle formations) or joints of the fetus that are telling of the fetal skeletal arrangement (e.g., a location of one or more of: the head, shoulder, hand, foot, knee, elbow, or the like). Sometimes relative locations of identified or inferred features can be used to infer the identities and/or relative locations of other features. For example, if the right shoulder of the fetus is turned outwards in a first direction relative to the fetus's head or torso, the left shoulder of the fetus (if not detectable) can be inferred to be turned outwards in a second (e.g., opposite) direction relative to the fetus's head or torso.

Using the information determined from the depth map, the controller 106 can be programmed or configured to identify detected portions 502 of the fetus, determine non-detected (e.g., missing or unidentifiable) portions 506 of the fetus, and can infer the non-detected portions 506 of the fetus based upon one or more computations (e.g., following one or more decision trees) as described herein and/or using techniques described in Shotton, Jamie, et al. “Real-time human pose recognition in parts from single depth images.” Communications of the ACM 56.1 (2013): 116-124, which is incorporated herein by reference in its entirety.

The controller 106 can determine a probable skeletal arrangement or a group of probable skeletal arrangements (e.g., multiple renderings of the fetus) based on the relative locations of the detected portions 502 of the fetus and inferred identities or locations of the non-detected portions 506 of the fetus. The controller 106 can also be programmed or configured to infer non-detected portions 506 of the fetus based on stored images or information related to previously detected or inferred skeletal arrangements for the fetus. For example, the controller 106 may receive a first signal set that shows strong signal performance for detecting or inferring features on a first side (e.g., right side) of the fetus at a first time, and at a second time, the controller 106 may receive a second signal set that demonstrates strong signal performance for detecting or inferring features on a second side (e.g., back, front, or left side) of the fetus but poor signal performance for detecting or inferring features on the first side of the fetus. Using the images or information associated with the first signal set in conjunction with the information associated with the second signal set, the controller 106 can be programmed or configured to infer non-detected features on the first side of the fetus and the second side of the fetus. In some embodiments, the controller 106 can combine portions of several images (e.g., selected views) of the fetus that are detected or computer generated at various times over a period to provide an image of the fetus having higher image quality than each of the separate images of the fetus.

In some embodiments, the controller 106 can be programmed or configured to identify at least one non-detected portion 506 of the fetus with an anatomical model (e.g., skeletal model, tissue model, joint connectivity model, or the like) that includes the non-detected portion 506 of the fetus and at least one detected portion 502 of the fetus. For example, the controller 106 can determine an anatomical structure associated with the non-detected portion 506 based upon an anatomical structure associated with the detected portion 502 of the fetus. In some embodiments, the anatomical model can be based upon a detected spatial parameter (e.g., length or width) associated with the detected portion 502 of the fetus, a development stage, a gender, or any other detected or specified information about the fetus. The controller 106 can also determine at least one of an identity, a position or an orientation of the non-detected portion 506 based upon at least one of a position or an orientation of the detected portion 502 and connectivity information (e.g., connectivity of the detected portion 502 and the non-detected portion 506) that is derived from the anatomical model. The identity, position, or orientation of the non-detected portion 506 of the fetus can also be based on a spatial relationship between at least two detected portions 502 of the fetus. For example, the controller 106 can infer that a non-detected portion is a knee and infer its location and/or the angle it is bent at based on detecting and identifying a corresponding foot and thigh of the fetus and determining the relative locations of the two features. In some embodiments, the controller 106 can be programmed or configured to create a computer-generated image of the non-detected portion 506 based upon the anatomical model.

In some embodiments, the controller 106 can be programmed or configured to identify at least one non-detected portion 506 of the fetus with a connectivity model that includes a predetermined connectivity of the non-detected portion 506 of the fetus and at least one detected portion 502 of the fetus. For example, the controller 106 can be programmed or configured to determine an anatomical structure associated with the non-detected portion 506 based upon a predetermined connectivity with another anatomical structure that is associated with the detected portion 502 of the fetus. In some embodiments, the connectivity model can include at least one of a hard tissue connectivity model or a soft tissue connectivity model. The connectivity model can be based upon a detected spatial parameter (e.g., length or width) associated with the detected portion 502 of the fetus, a development stage, a gender, or any other detected or specified information about the fetus. The controller 106 can also be programmed or configured to determine at least one of a position or an orientation of the non-detected portion 506 based upon at least one of a position or an orientation of the detected portion 502 and the predetermined connectivity information of the connectivity model. For example, the predetermined connectivity information can include one or more stored data or images of postures, tissue alignments, or the like. In some embodiments, the controller 106 can be programmed or configured to determine at least one of a position or an orientation of the non-detected portion 506 based upon at least one of a position or an orientation of a first detected portion 502 and at least a second detected portion 502 and predetermined connectivity information associated with the non-detected portion 506 and the first and second detected portions 502 of the fetus. In some embodiments, the controller 106 can also be programmed or configured to create a computer-generated image of the non-detected portion 506 based upon a comparison of the determined image and information associated with the connectivity model. In some embodiments, the connectivity model is based upon an anatomical model that characterizes at least one of appearance, relative size attributes, or connectivity of fetal tissue structures.

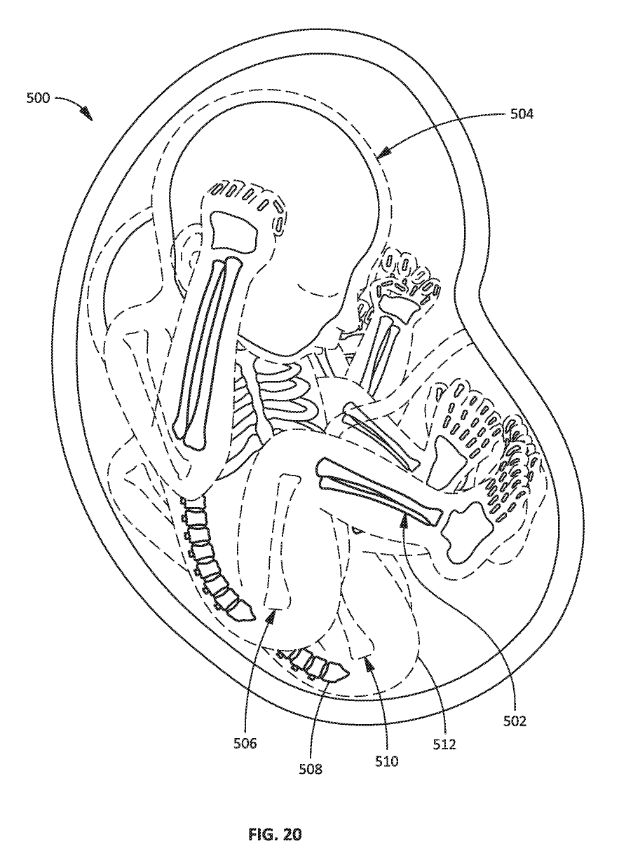

As shown in FIG. 20, the fetal imaging device 100 can be used to image multiple fetuses within the body 200. In this regard, the controller 106 can be further configured to identify or image one or more portions of at least a second fetus. For example, the controller 106 can be programmed or configured to receive an ultrasound image or create a computer-generated image of one or more detected portions 508 of the second fetus. In some embodiments, the controller 106 can be programmed or configured to apply a texture map 512 for the second fetus. The texture map 512 can be based upon the texture map 504 applied for the first fetus. For example, the second texture map 512 can be identical or based upon the same or similar criteria. In some embodiments, the second texture map 512 can differ from the first texture map 504. For example, the second texture map 512 can be associated with a different gender or different relative. By way of further example, the first texture map 504 can include paternal features, and the second texture map 512 can include maternal features. The controller 106 can be programmed or configured to map a facial expression onto the image of the second fetus.

In some embodiments, the controller 106 can be programmed or configured to identify at least one non-detected portion 510 of the second fetus with an anatomical model that includes the non-detected portion 510 of the second fetus and at least one detected portion 508 of the second fetus. For example, the controller 106 can be programmed or configured to determine an anatomical structure associated with the non-detected portion 510 based upon an anatomical structure associated with the detected portion 508 of the second fetus. This can be the same anatomical model as used for the first fetus or a second anatomical model based upon one or more attributes of the second fetus. In some embodiments, the anatomical model can be based upon a detected spatial parameter (e.g., length or width) associated with the detected portion 508 of the second fetus, a development stage, a gender, or any other detected or specified information about the second fetus. The controller 106 can also be programmed or configured to determine at least one of a position or an orientation of the non-detected portion 510 based upon at least one of a position or an orientation of the detected portion 508 and connectivity information (e.g., connectivity of the detected portion 508 and the non-detected portion 510) that is derived from the anatomical model. In some embodiments, the controller 106 can be programmed or configured to create a computer-generated image of the non-detected portion 510 of the second fetus based upon the anatomical model.

In some embodiments, the controller 106 can be programmed or configured to identify at least one non-detected portion 510 of the second fetus with a connectivity model that includes a predetermined connectivity of the non-detected portion 510 of the second fetus and at least one detected portion 508 of the second fetus. For example, the controller 106 can be programmed or configured to determine an anatomical structure associated with the non-detected portion 510 based upon a predetermined connectivity with another anatomical structure that is associated with the detected portion 508 of the second fetus. In some embodiments, the connectivity model can include at least one of a hard tissue connectivity model or a soft tissue connectivity model. This can be the same connectivity model as used for the first fetus or a second connectivity model based upon one or more attributes of the second fetus. In some embodiments, the connectivity model can be based upon a detected spatial parameter (e.g., length or width) associated with the detected portion 508 of the second fetus, a development stage, a gender, or any other detected or specified information about the second fetus. The controller 106 can also be programmed or configured to determine at least one of a position or an orientation of the non-detected portion 510 based upon at least one of a position or an orientation of the detected portion 508 and the predetermined connectivity information of the connectivity model. For example, the predetermined connectivity information can include one or more stored postures, tissue alignments, or the like. In some embodiments, the controller 106 can be programmed or configured to determine at least one of a position or an orientation of the non-detected portion 510 based upon at least one of a position or an orientation of a first detected portion 508 and at least a second detected portion 508 and predetermined connectivity information associated with the non-detected portion 510 and the first and second detected portions 508 of the second fetus. In some embodiments, the controller 106 can be programmed or configured to create a computer-generated image of the non-detected portion 510 based upon the connectivity model. In some embodiments, the connectivity model is based upon an anatomical model that characterizes at least one of appearance, relative size attributes, or connectivity of fetal tissue structures.

In some embodiments, the controller 106 can be programmed or configured to identify the non-detected portion 510 of the second fetus based upon the one or more detected portions 502 of the first fetus. The controller 106 can also be programmed or configured to determine at least one of a position or an orientation of the non-detected portion 510 of the second fetus based upon one or more detected portions 502 of the first fetus. For example, the positioning of the first fetus can affect the positioning of the second fetus within the body 200. In some embodiments, a combined connectivity model can include positioning of one or more portions of a first fetus relative to one or more portions of a second fetus within a body. The controller 106 can be programmed or configured to identify or determine spatial information for the non-detected portion 510 of the second fetus based upon a combined connectivity model and the one or more detected portions 502 of the first fetus. The controller 106 can also be programmed or configured to identify or determine spatial information for the non-detected portion 510 of the second fetus based upon a combined connectivity model, the one or more detected portions 508 of the second fetus, and the one or more detected portions 502 of the first fetus.

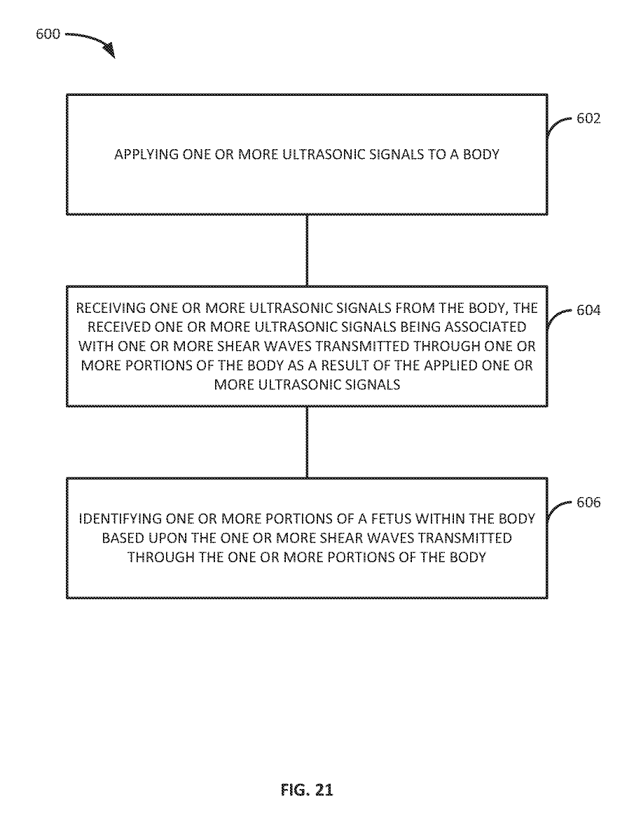

FIGS. 21-44 show various embodiments of a method 600 of fetal imaging. The method 600 can be performed by one or more components of the fetal imaging device 100 described herein or any other device (e.g., remote device 400) configured to support the operations described herein. At block 602, one or more ultrasonic signals are applied to a body. For example, one or more ultrasonic signals can be applied with at least one ultrasound element (e.g., ultrasound element 102). At block 604, one or more ultrasonic signals are received from the body. The one or more received ultrasonic signals can be associated with one or more shear waves transmitted through one or more portions of the body as a result of the one or more applied ultrasonic signals. For example, the one or more received ultrasonic signals can be received by at least one ultrasound element (e.g., ultrasound element 102) configured for shear wave elastography. At block 606, one or more portions of a fetus within the body can be identified based upon the one or more shear waves transmitted through the one or more portions of the body. For example, a controller (e.g., controller 106) can identify the one or more portions of the fetus.

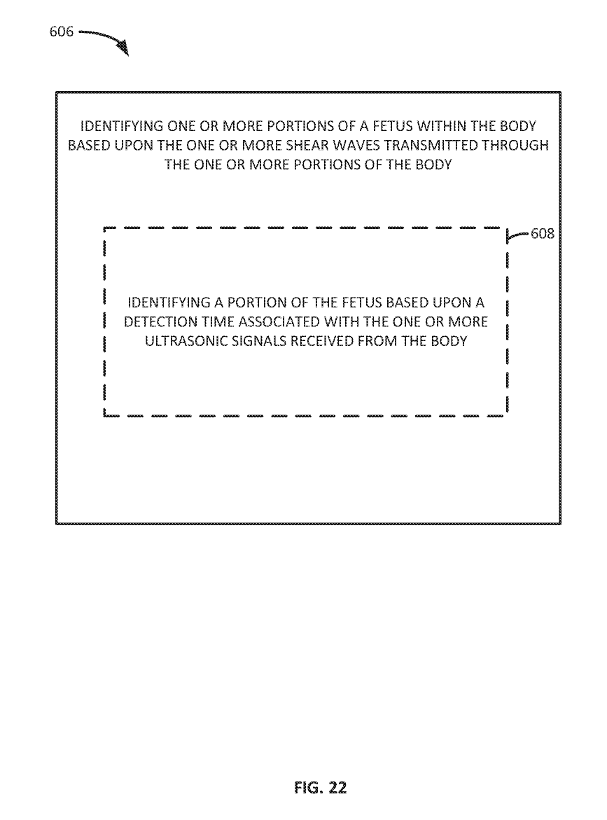

As shown in FIG. 22, block 606 can include identifying a portion of the fetus based upon a detection time associated with the one or more ultrasonic signals received from the body (block 608). For example, the controller can perform a time gating analysis to identify or image the one or more portions of the fetus. In some embodiments, the controller can isolate signals corresponding to fetal portions or filter out signals corresponding to non-fetal portions of the body.

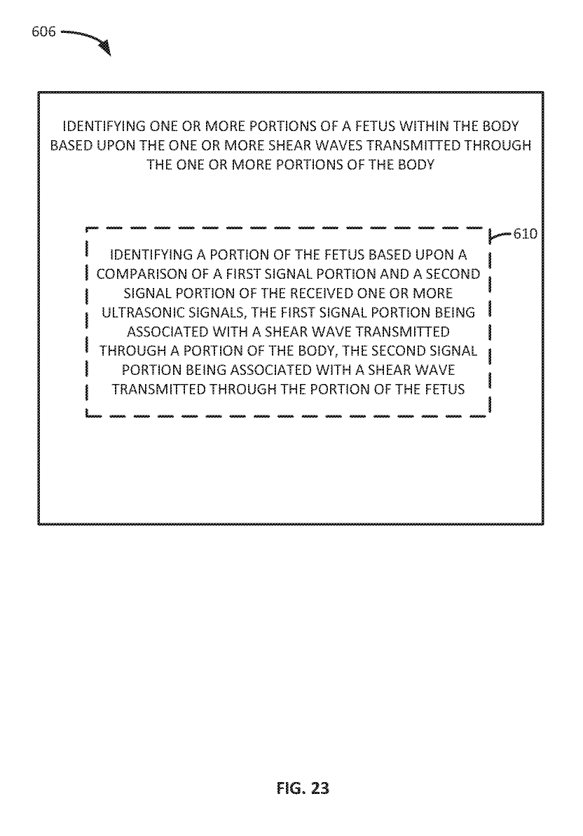

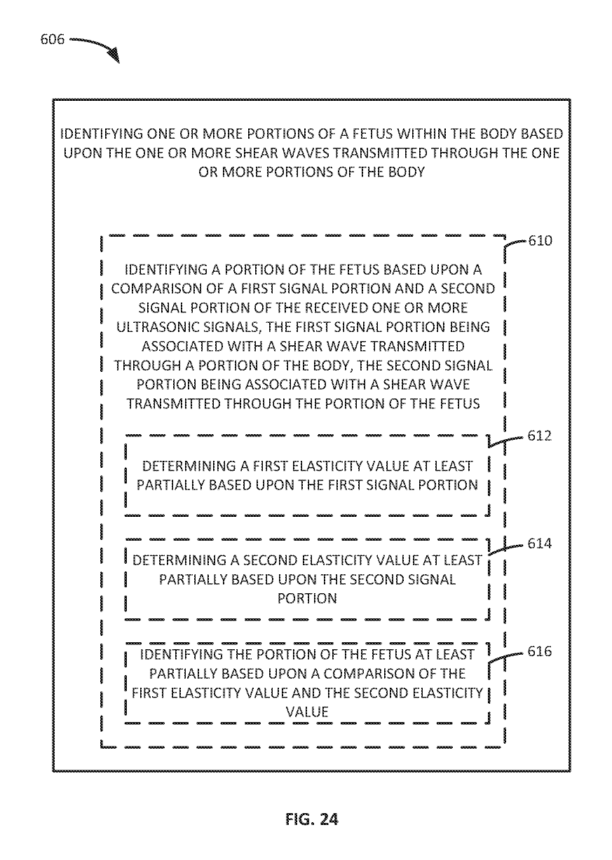

As shown in FIG. 23, block 606 can include identifying a portion of the fetus based upon a comparison of a first signal portion and a second signal portion of the received one or more ultrasonic signals (block 610). The first signal portion can be associated with a shear wave transmitted through a portion of the body, and the second signal portion can be associated with a shear wave transmitted through the portion of the fetus. In some embodiments, the controller can perform a comparison between one or more attributes of the first signal portion and one or more attributes of the second signal portion. For example, the controller compares a frequency, amplitude, velocity, phase, scattering value, reflectance value, or other linear/non-linear attribute of the first signal portion to a frequency, amplitude, velocity, phase, scattering value, reflectance value, or other linear/non-linear attribute of the second signal portion. As shown in FIG. 24, block 610 can also include: determining a first elasticity value at least partially based upon the first signal portion (block 612); determining a second elasticity value at least partially based upon the second signal portion (block 614); and identifying the portion of the fetus at least partially based upon a comparison of the first elasticity value and the second elasticity value (block 616).

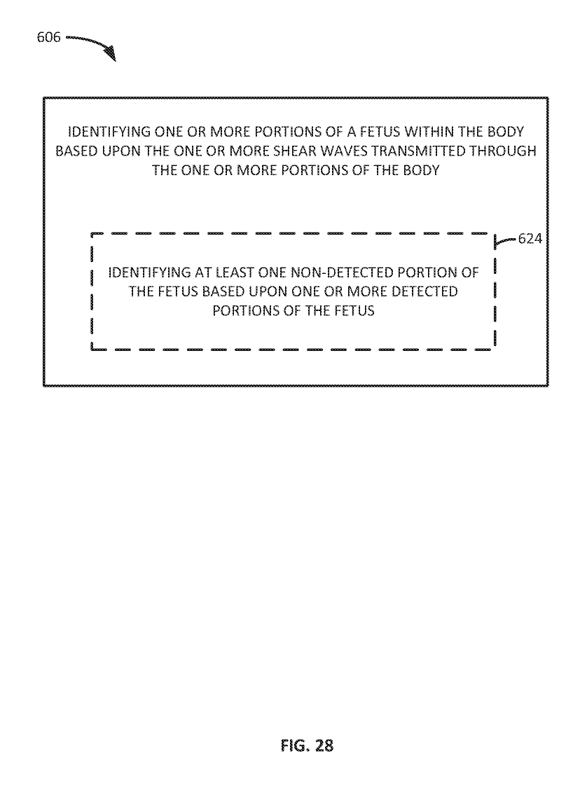

As shown in FIG. 28, block 606 can include identifying at least one non-detected portion of the fetus based upon one or more detected portions of the fetus (block 624). In some embodiments, the non-detected portion of the fetus can be identified based upon an anatomical model or a connectivity model including connectivity information for the one or more detected portions of the fetus and the non-detected portion of the fetus. In some embodiments, an alternative imaging angle can be determined for imaging the non-detected portion. The non-detected portion can be imaged from an alternative imaging angle with a second ultrasound element or by repositioning the same ultrasound element.

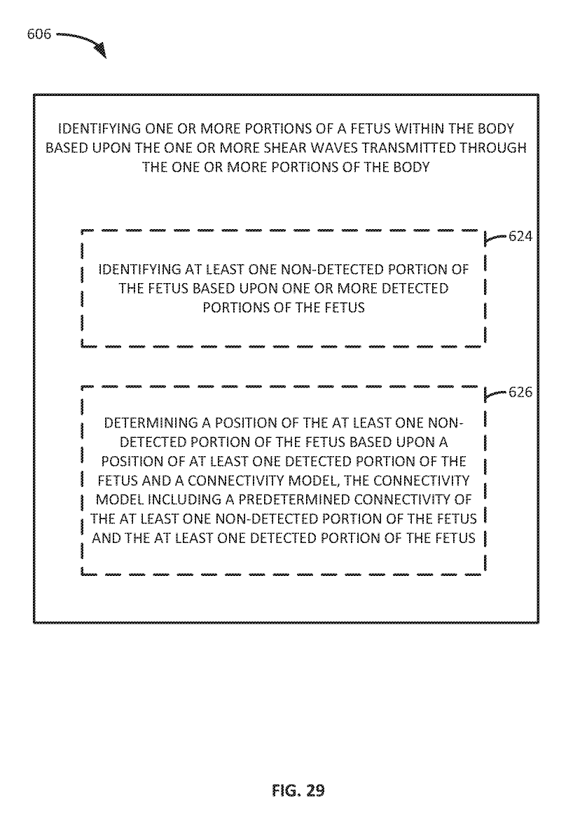

As shown in FIG. 29, block 606 can also include determining a position of the non-detected portion of the fetus based upon a position of at least one detected portion of the fetus and a connectivity model that includes a predetermined connectivity of the detected and non-detected portions of the fetus (block 626).

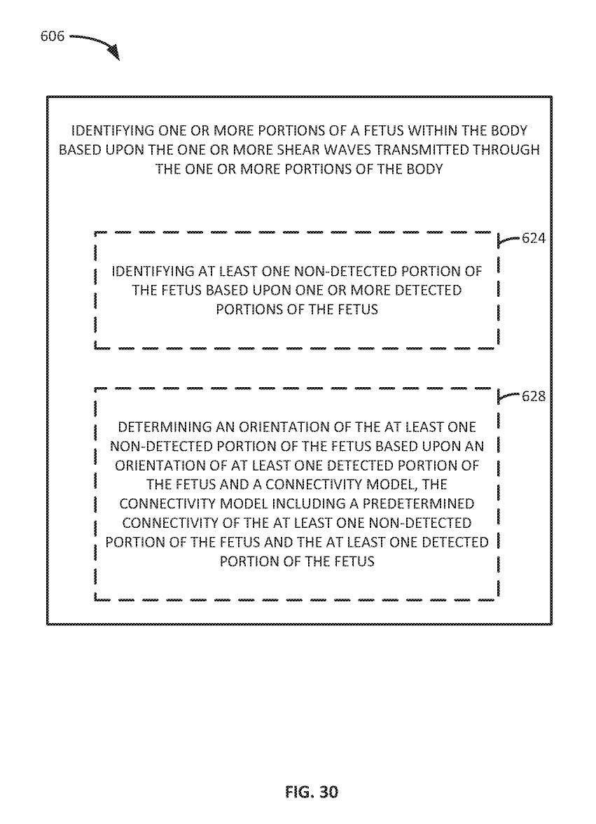

As shown in FIG. 30, block 606 can also include determining an orientation of the non-detected portion of the fetus based upon an orientation of at least one detected portion of the fetus and a connectivity model that includes a predetermined connectivity of the detected and non-detected portions of the fetus (block 628).

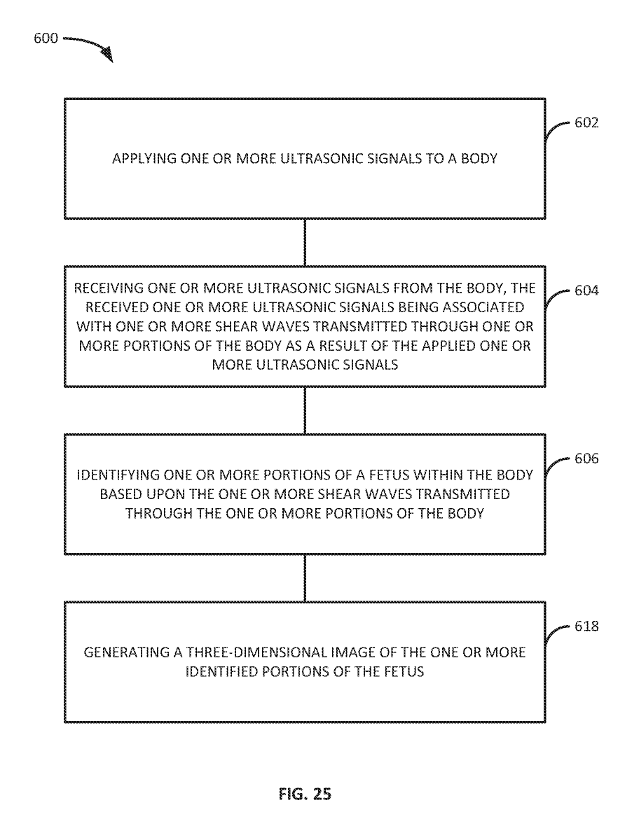

Referring to FIG. 25, a three-dimensional image can be generated based upon the one or more identified portions of the fetus (block 618). For example, the controller can create a three-dimensional graphical rendering based upon the one or more received ultrasonic signals.

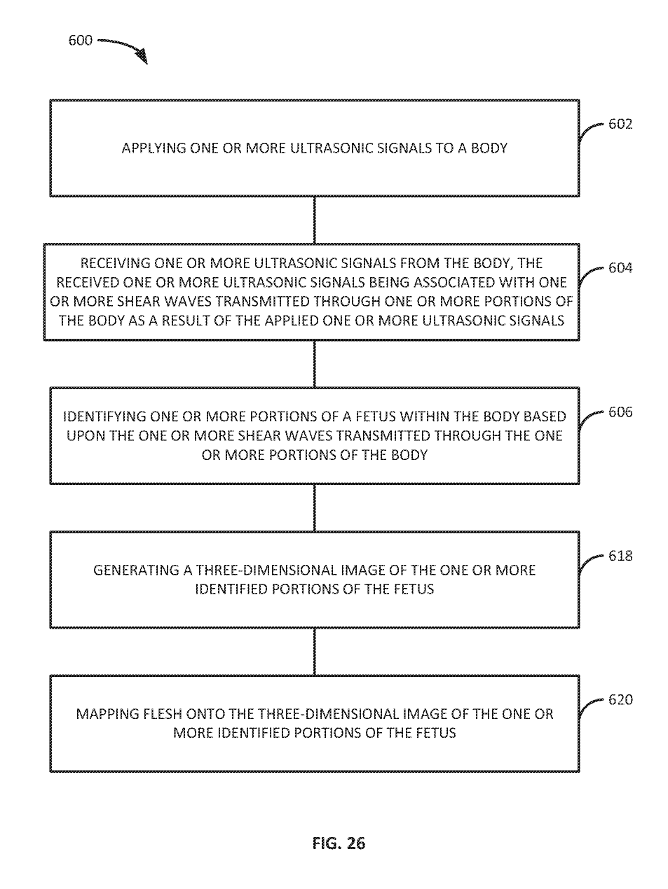

As shown in FIG. 26, the method 600 can further include mapping flesh onto the three-dimensional image of the one or more identified portions of the fetus (block 620). For example, the controller can apply a texture map to the three-dimensional graphical rendering of the fetus. By way of further example, the controller can infer and apply a texture map based upon a predetermined or stored texture map, a genomic element, a skin tone, a demographic, a stage of development, an image of a person, or the like.

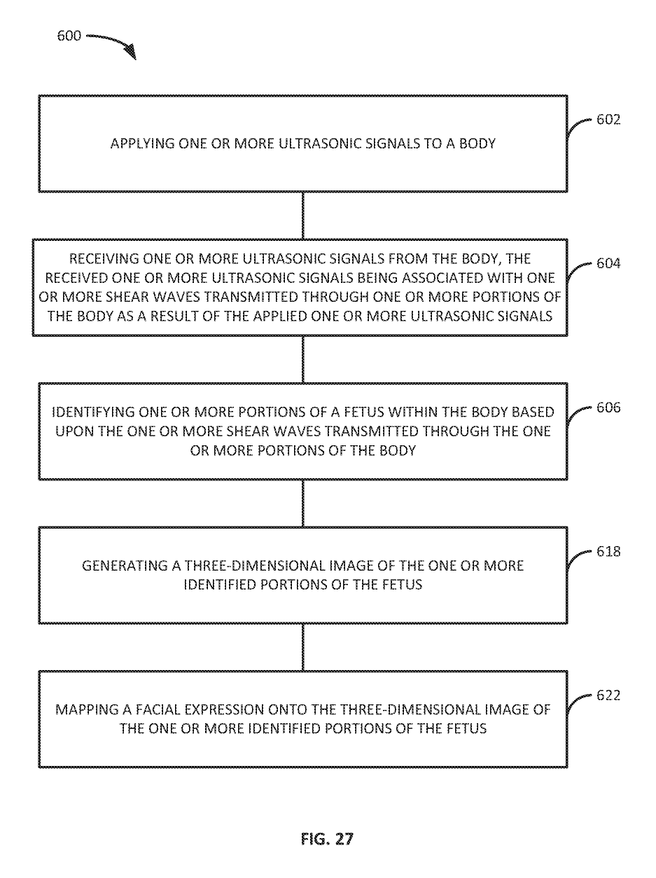

As shown in FIG. 27, the method 600 can further include mapping a facial expression onto the three-dimensional image of the one or more identified portions of the fetus (block 622). For example, the controller can map a facial expression onto the three-dimensional graphical rendering of the fetus. By way of further example, the controller can map a facial expression that is randomly generated or based upon a time, a date, a detected sound, a detected motion, a detected temperature, an identified fetal posture, a user input, or the like.

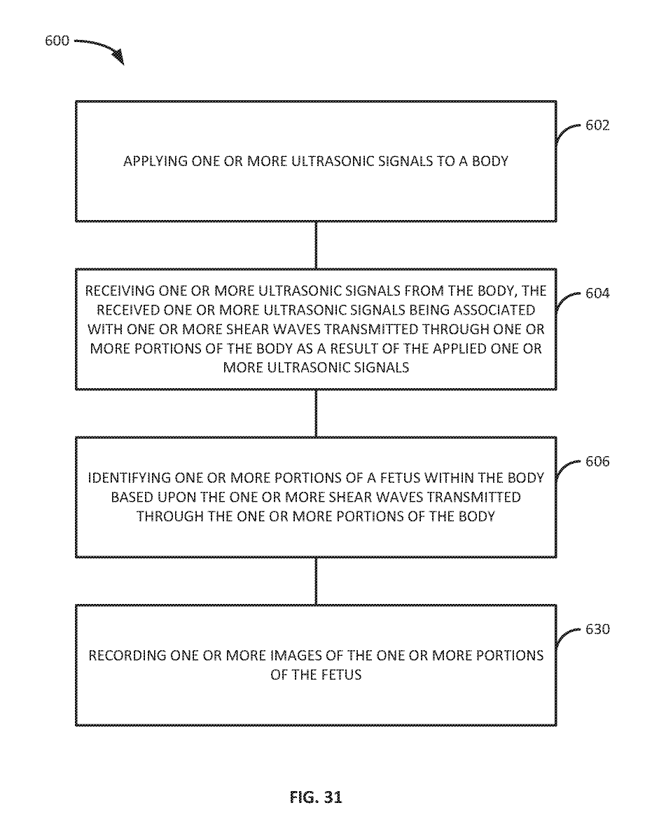

Referring to FIG. 31, one or more images (e.g., ultrasound images or computer-generated images) of the one or more portions of the fetus can be recorded (block 630). For example, the controller can record an image based upon the one or more received ultrasonic signals. In some embodiments, the one or more images are recorded periodically, according to a schedule, or in response to a user input, a request from a second device, or detection of fetal activity above or below a threshold fetal activity.

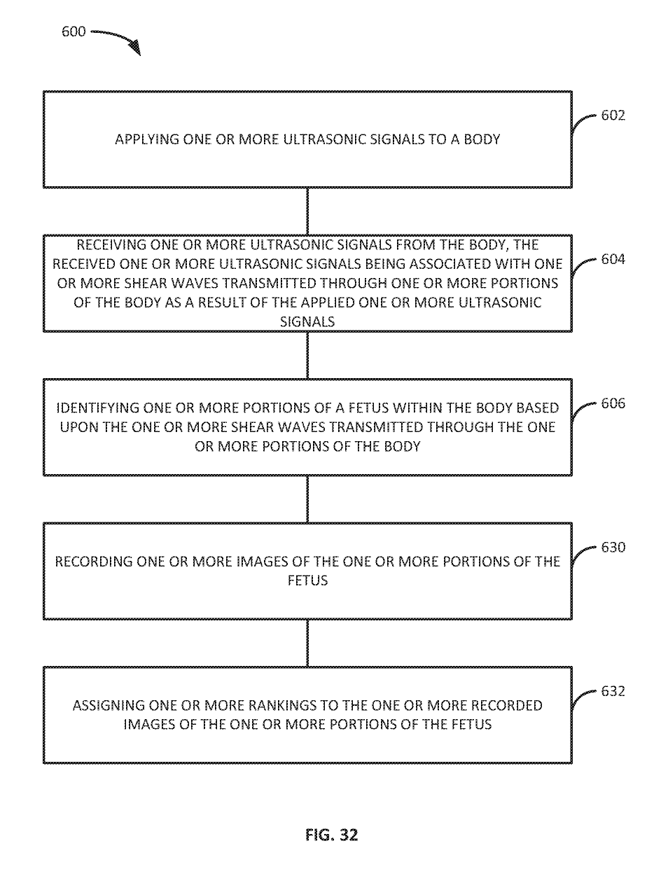

As shown in FIG. 32, the method 600 can further include assigning one or more rankings to the one or more recorded images of the one or more portions of the fetus (block 632). For example, the controller can assign a score or ranking to a recorded image of the fetus based upon an image quality or other characteristic of the image.

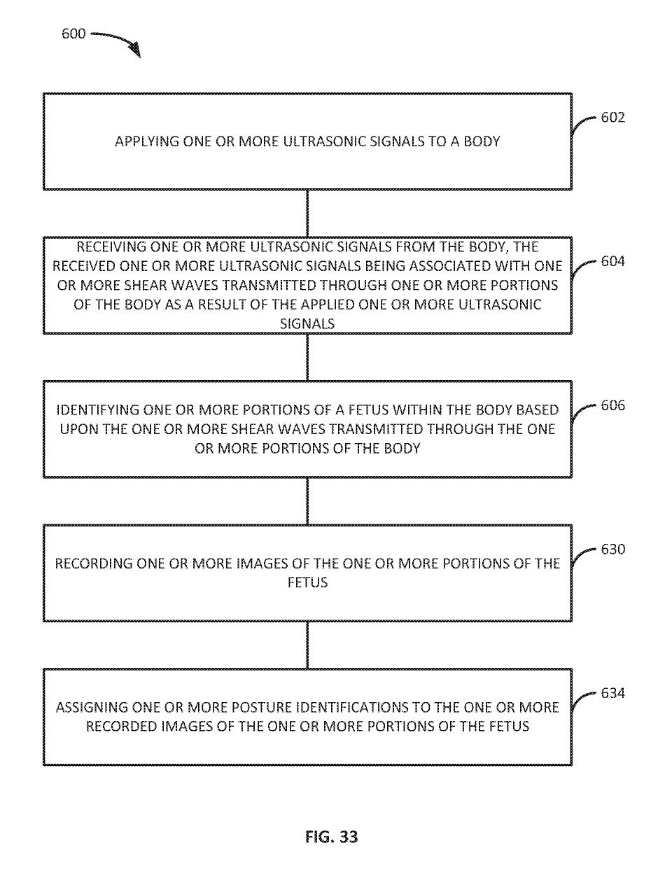

As shown in FIG. 33, the method 600 can further include assigning one or more posture identifications to the one or more recorded images of the one or more portions of the fetus (block 634). For example, the controller can identify a posture of the fetus based upon connectivity data associated with the one or more identified portions of the fetus or based upon a comparison between the recorded image and a previously stored image having an identified posture.

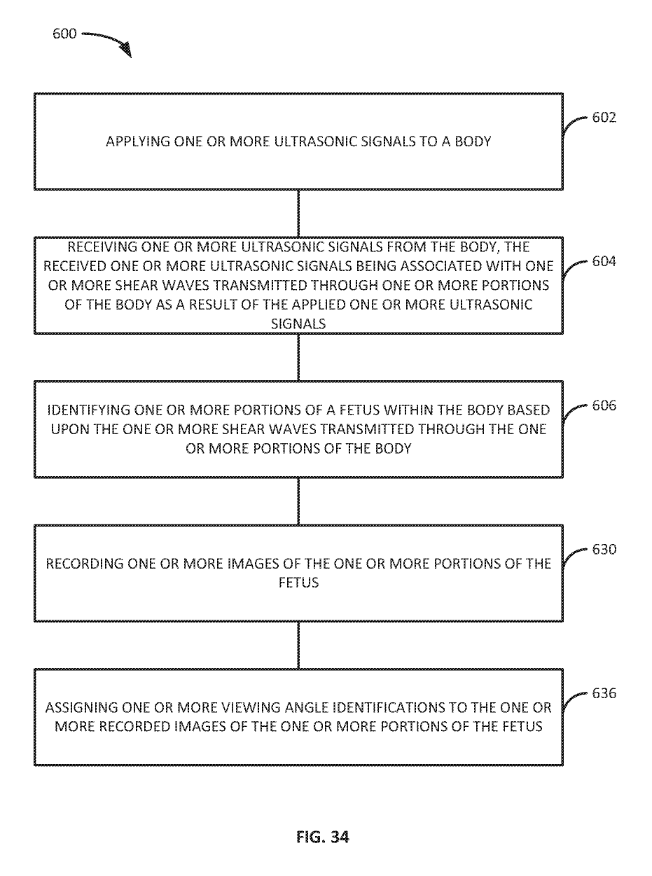

As shown in FIG. 34, the method 600 can further include assigning one or more viewing angle identifications to the one or more recorded images of the one or more portions of the fetus (block 636). For example, the controller can assign a viewing angle to an image of the fetus based upon a predetermined viewing angle associated with an ultrasound element, or the viewing angle can be derived from the image of the fetus.

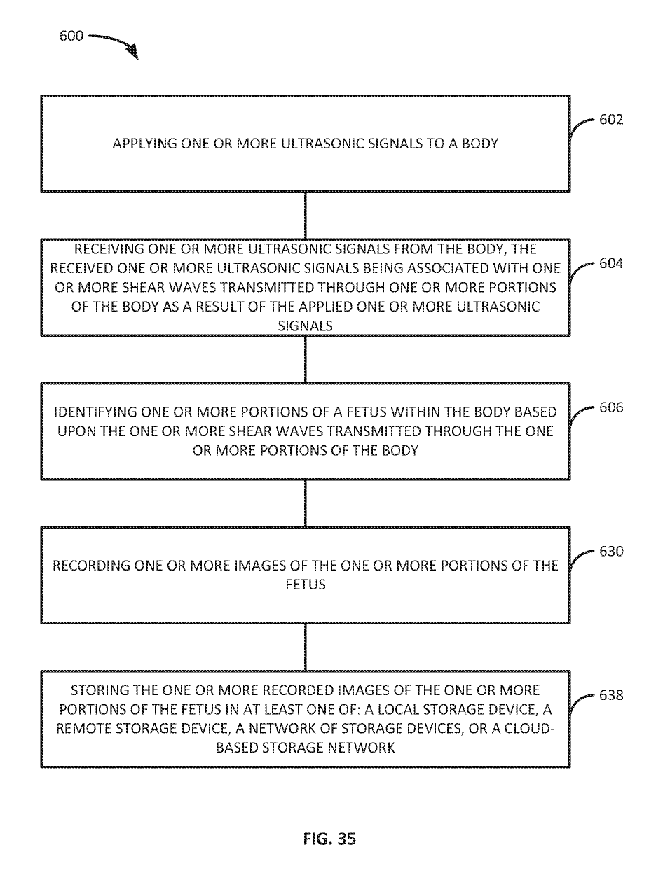

As shown in FIG. 35, the method 600 can further include storing the one or more recorded images of the one or more portions of the fetus in at least one of a local storage device, a remote storage device, a network of storage devices, or a cloud-based storage network (block 638). For example, the controller can store the one or more recorded images to a storage device (e.g., storage device 136).

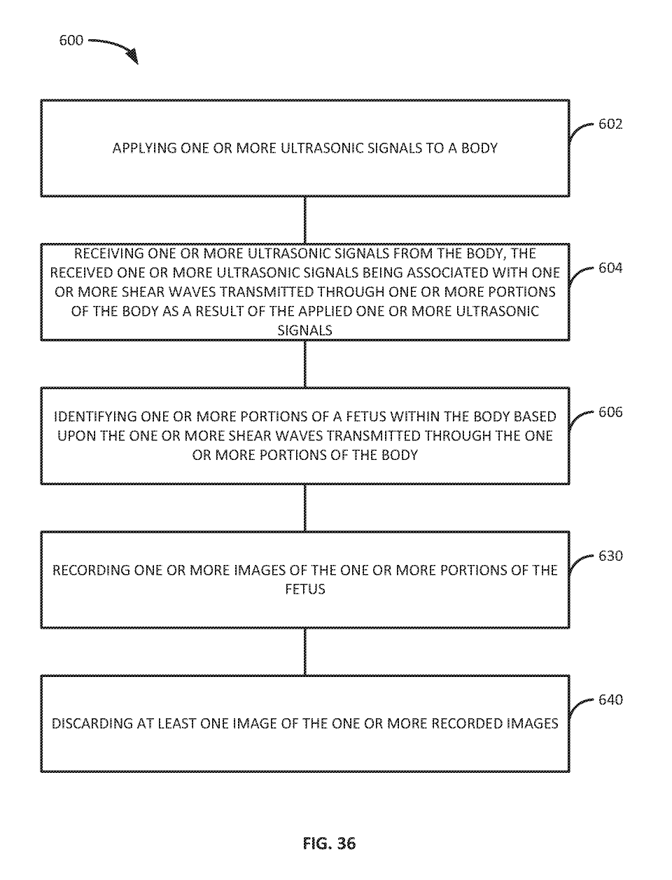

As shown in FIG. 36, the method 600 can further include discarding at least one image of the one or more recorded images (block 640). For example, the controller can delete an image from the storage device to increase available storage capacity or remove undesirable images. In some embodiments, an image can be discarded based upon detecting at least one of a redundant viewing angle, a redundant fetal posture, an obscured portion of the fetus, or an expired timestamp.

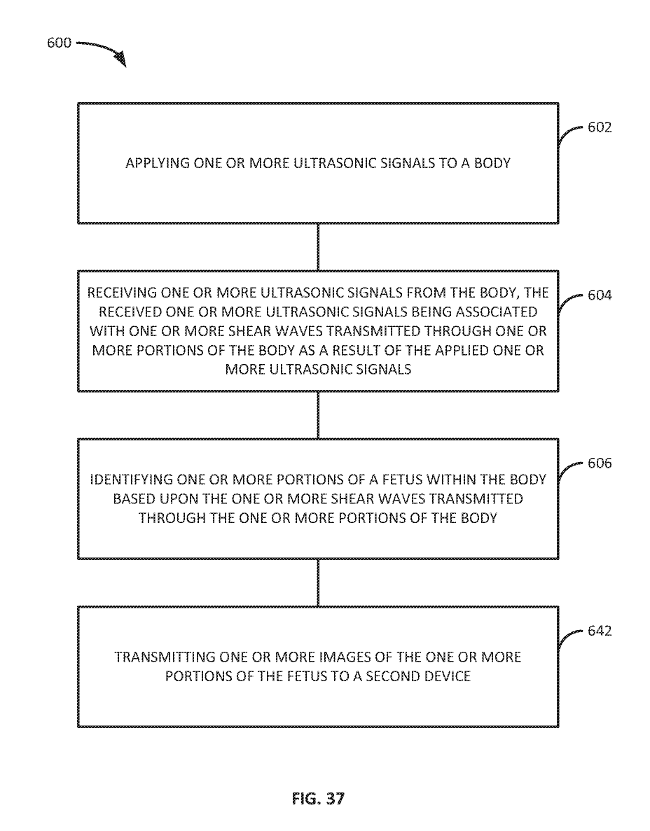

Referring to FIG. 37, one or more images (e.g., ultrasound images or computer-generated images) of the one or more portions of the fetus can be transmitted to a second device (block 642). For example, a transmitter (e.g., transmitter 144) can transmit the one or more images of the fetus to a second device (e.g., remote device 400). In some embodiments, the one or more images are streamed in real time or transmitted periodically, according to a schedule, or in response to a user input, a request from a second device, or detection of fetal activity above or below a threshold fetal activity.

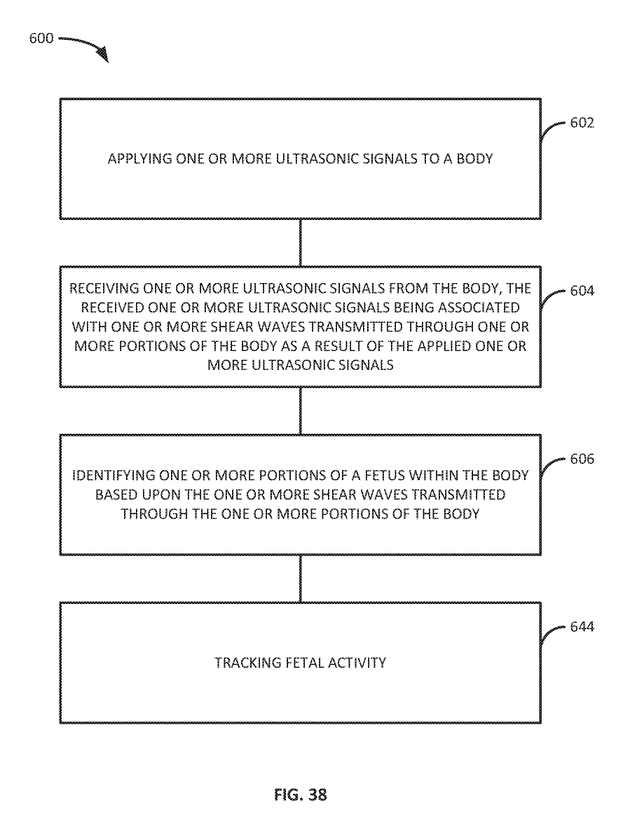

Referring to FIG. 38, an activity level of the fetus can be tracked (block 644). In some embodiments, the controller can track fetal activity by identifying a difference between a first image of the fetus and a second image of the fetus. In some embodiments, fetal activity can be detected with an acoustic sensor (e.g., sensor 130) or a contact force sensor (e.g., sensor 132) configured to detect vibrations or impulses associated with fetal activity (e.g., kicking or other movements of the fetus).

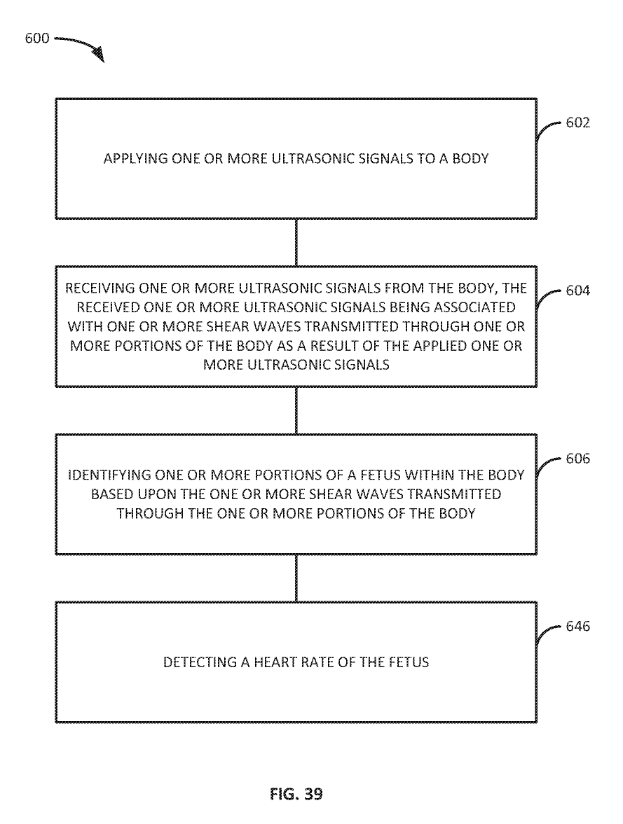

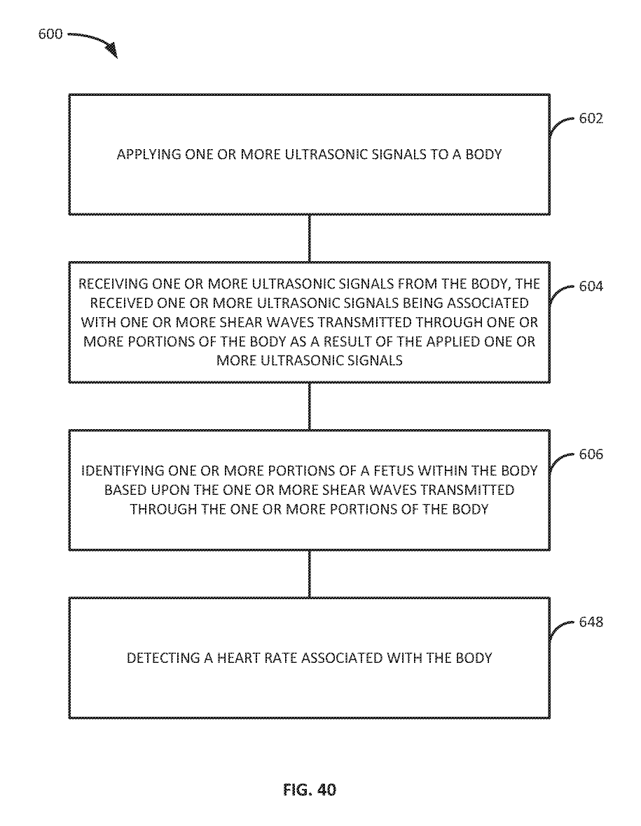

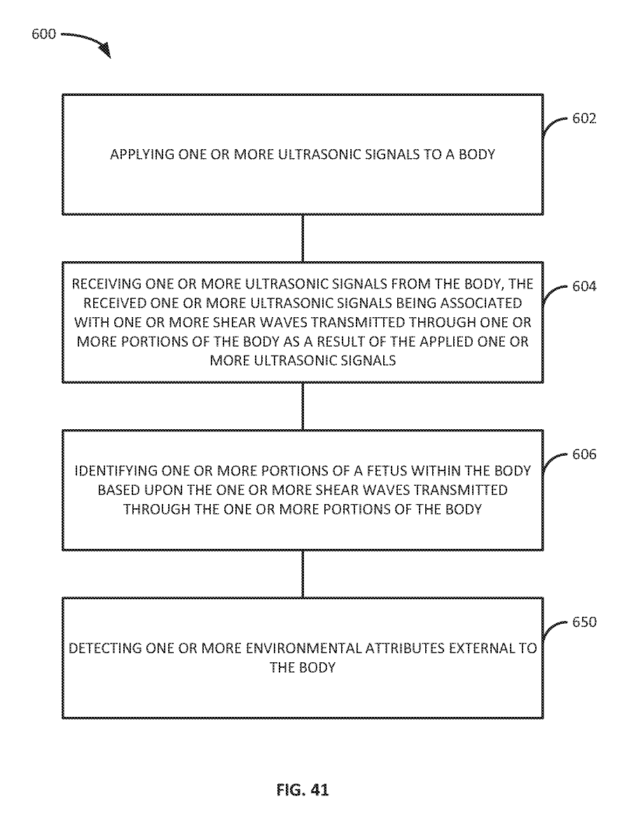

In some embodiments, various environmental or physiological attributes associated with the fetus or a body (e.g., body 200) bearing the fetus can be monitored. As shown in FIG. 39, a heart rate of the fetus can be detected (block 646). For example, the heart rate of the fetus can be detected with an appropriate physiological sensor (e.g., sensor 126). As shown in FIG. 40, a non-fetal heart rate associated with the body bearing the fetus can be detected (block 648). For example, the non-fetal heart rate can be detected with an appropriate physiological sensor (e.g., sensor 128). As shown in FIG. 41, one or more environmental attributes external to the body can be detected (block 650). For example, the one or more environmental attributes can be detected with one or more environmental sensors (e.g., sensor 134).

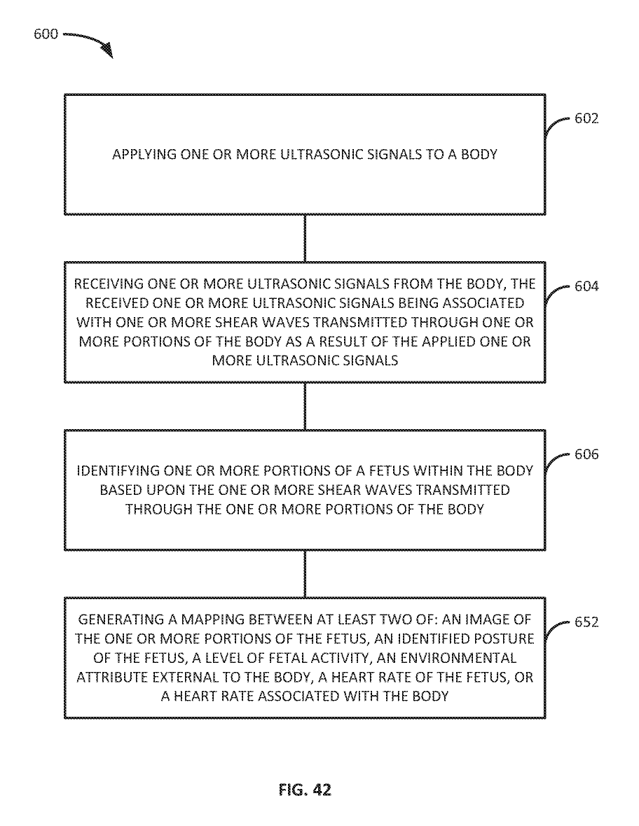

Referring to FIG. 42, a mapping can be established between at least two of: an image of the one or more portions of the fetus, an identified posture of the fetus, a level of fetal activity, an environmental attribute external to the body, a heart rate of the fetus, or a heart rate associated with the body (block 652). For example, the controller can generate a one-to-one or time-indexed mapping that shows a correlation between a first imaging data or sensor data value and a second imaging data or sensor data value.

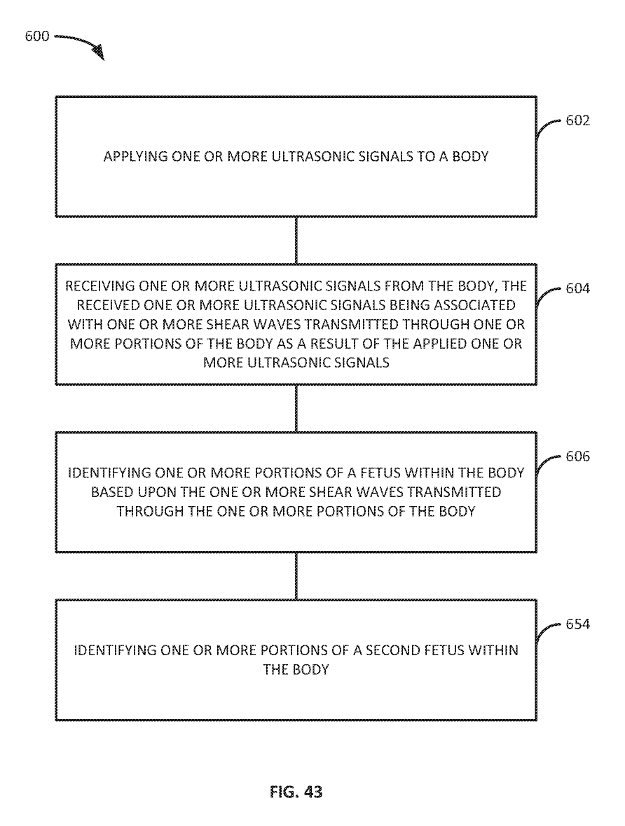

Referring to FIG. 43, one or more portions of a second fetus within the body can be identified (block 654). For example, the controller can identify one or more portions of the second fetus based upon the one or more received ultrasonic signals. In some embodiments, one or more operations described herein for the first fetus can be performed for the second fetus. In some embodiments, identification of one or more portions of the second fetus or determination of positioning or connectivity information can be based upon the first fetus. For example, the controller can identify one or more portions of the second fetus based upon one or more identified portions of the first fetus.

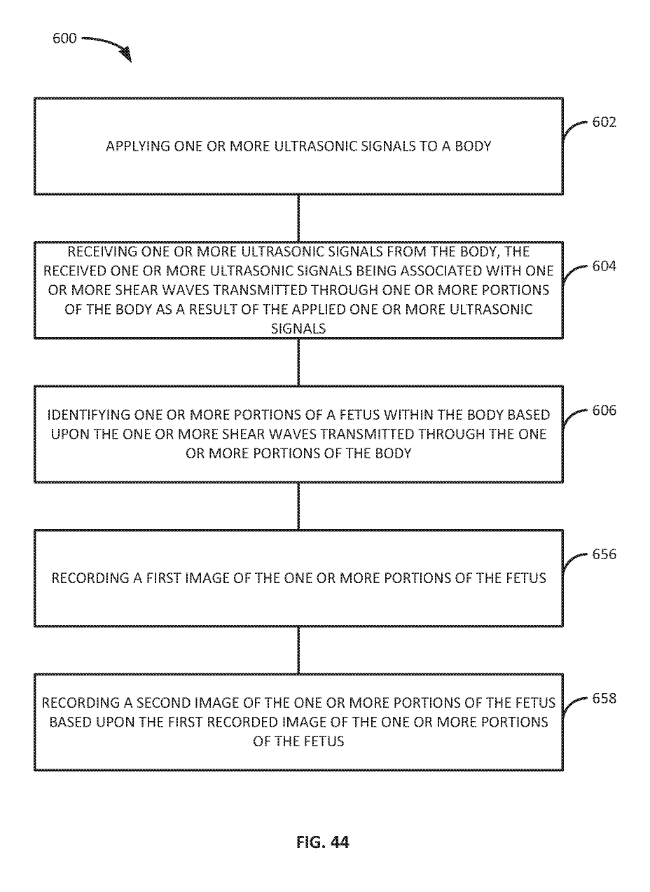

An embodiment of method 600 is shown in FIG. 44 to include recording a first image of the one or more portions of the fetus (block 656) and recording a second image of the one or more portions of the fetus based upon the first recorded image of the one or more portions of the fetus (block 658). For example, a second image can be recorded or collected because of an identified posture or viewing angle associated with the first image or lack thereof. In some embodiments, the second image can be recorded or collected because the first image includes a non-identifiable or obscured portion of the fetus. In some embodiments, the second image can be recorded or collected because of a score or ranking (e.g., computer-generated or user-driven score or ranking) assigned to the first image.

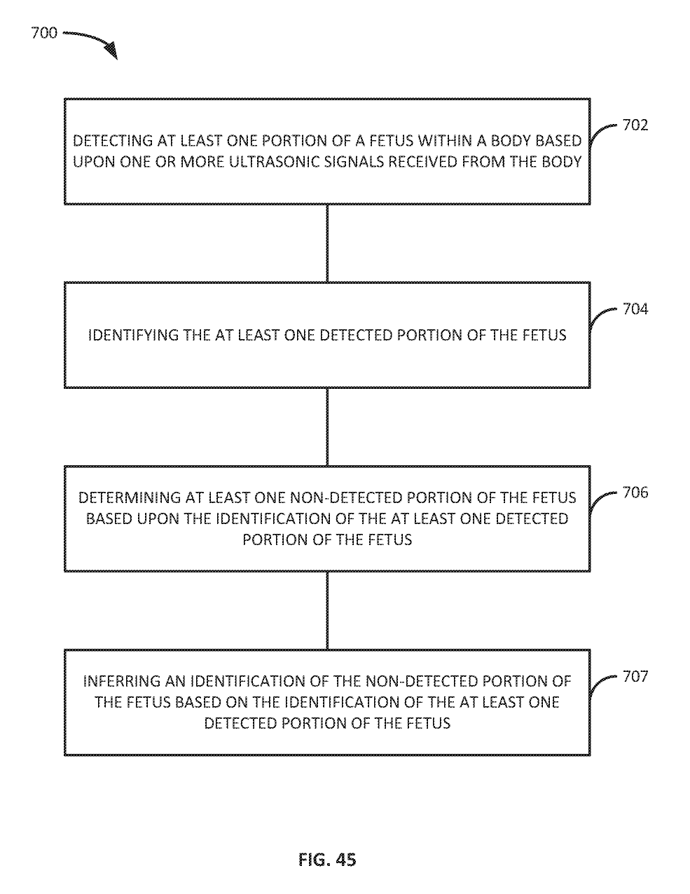

FIGS. 45-59 show various embodiments of a method 700 of fetal imaging. The method 700 can be performed by one or more components of the fetal imaging device 100 described herein or any other device (e.g., remote device 400) configured to support the operations described herein. At block 702, one or more portions of a fetus within a body can be detected based upon one or more ultrasonic signals received from the body. For example, the one or more received ultrasonic signals can be received by at least one ultrasound element (e.g., ultrasound element 102) configured for ultrasonography or shear wave elastography. At block 704, one or more portions of a fetus within the body can be identified. For example, a controller (e.g., controller 106) can identify the one or more portions of the fetus. The one or more portions can simply be identified as belonging to the fetus or can be further identified as including one or more particular anatomical structures of the fetus. In some instances, one or more portions of the fetus are not detected. For example, the one or more non-detected portions of the fetus can include a portion of the fetus that is obscured, only partly detected, or detected at a low resolution. At block 706, at least one non-detected portion of the fetus can be determined based upon at least one detected portion of the fetus. For example, a portion of the fetus can be determined to be non-detected when an expected portion of the fetus appears to be missing or obscured. The non-detected portion can be determined based upon one or more detected portions using process of elimination or by comparing the detected portions to an anatomical model (e.g., skeletal or tissue model) or a connectivity (e.g., joint connectivity) model. At block 707, identities, locations, and/or orientations of the non-detected portion or portions can be inferred based on the identification (e.g., identity, location, orientation, etc.) of the detected portions of the fetus. In some embodiments, the non-detected portion of the fetus can be identified based upon an anatomical model or a connectivity model including connectivity information for the detected portion of the fetus and the non-detected portion of the fetus. For example, a depth map can be computed based on the detected portions of the fetus, and the depth map can be compared to one or more anatomical models and/or connectivity models to infer attributes (e.g., identity, size/dimensions, position, orientation, etc.) of the non-detected portion or portions of the fetus.

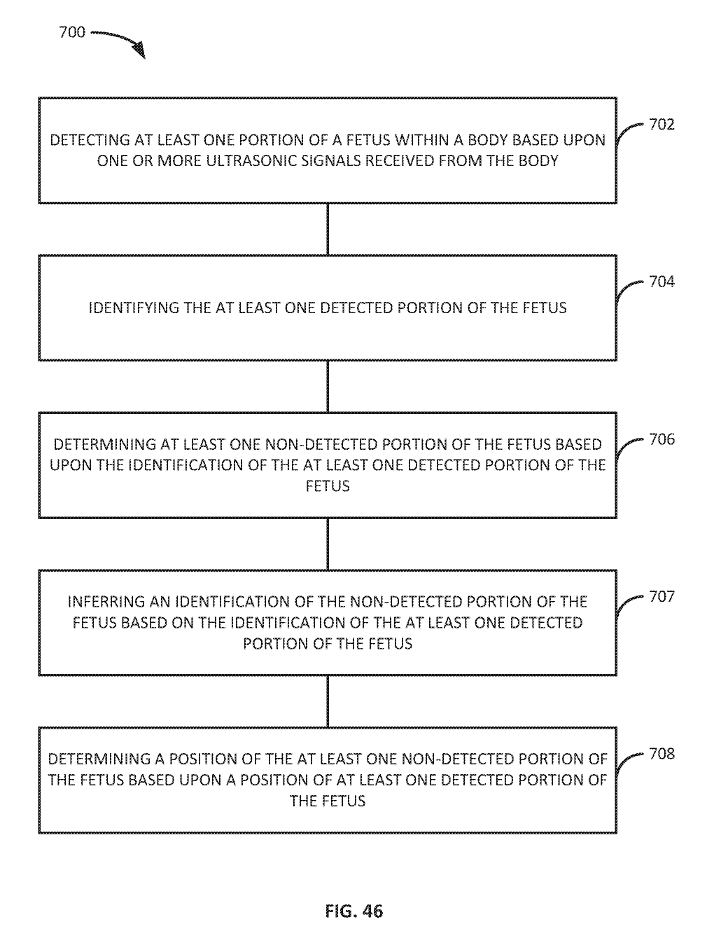

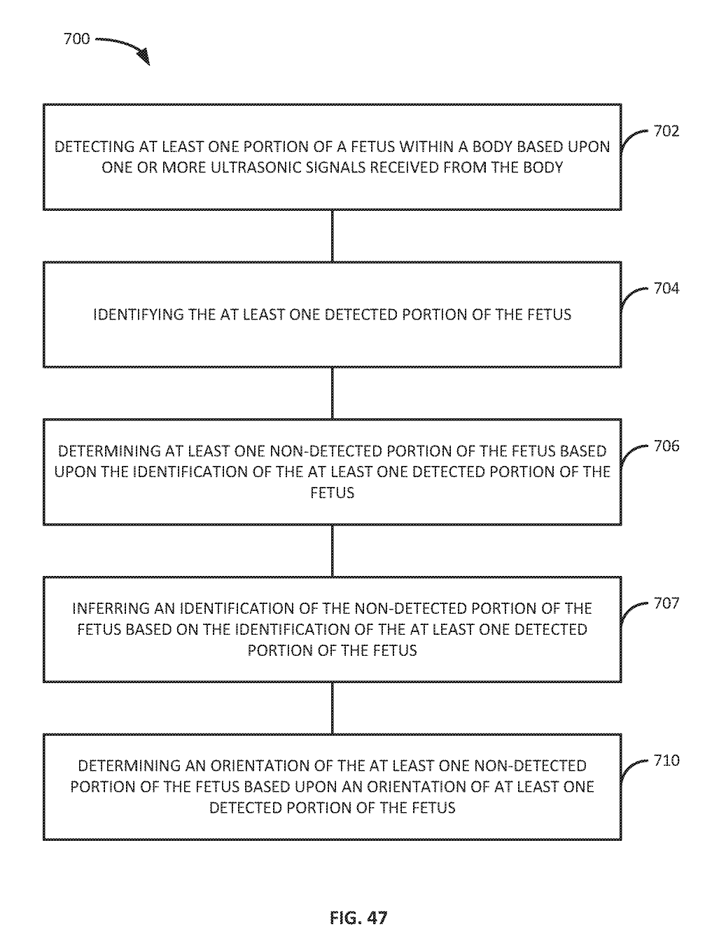

Referring to FIG. 46, a position of the non-detected portion of the fetus can be determined based upon a position of the detected portion of the fetus (block 708). For example, the position can be determined using an anatomical model or a connectivity model that includes relative positions of the detected and non-detected portions of the fetus. Referring to FIG. 47, an orientation of the non-detected portion of the fetus can be determined based upon an orientation of the detected portion of the fetus (block 710). For example, the orientation can be determined using an anatomical model or a connectivity model that includes relative orientations of the detected and non-detected portions of the fetus. In some embodiments, a connectivity model further includes information associated with possible postures of the fetus. In some embodiments, at least one of the position or the orientation of the non-detected portion of the fetus can be determined based upon a tissue connectivity associated with an identified posture of the fetus.

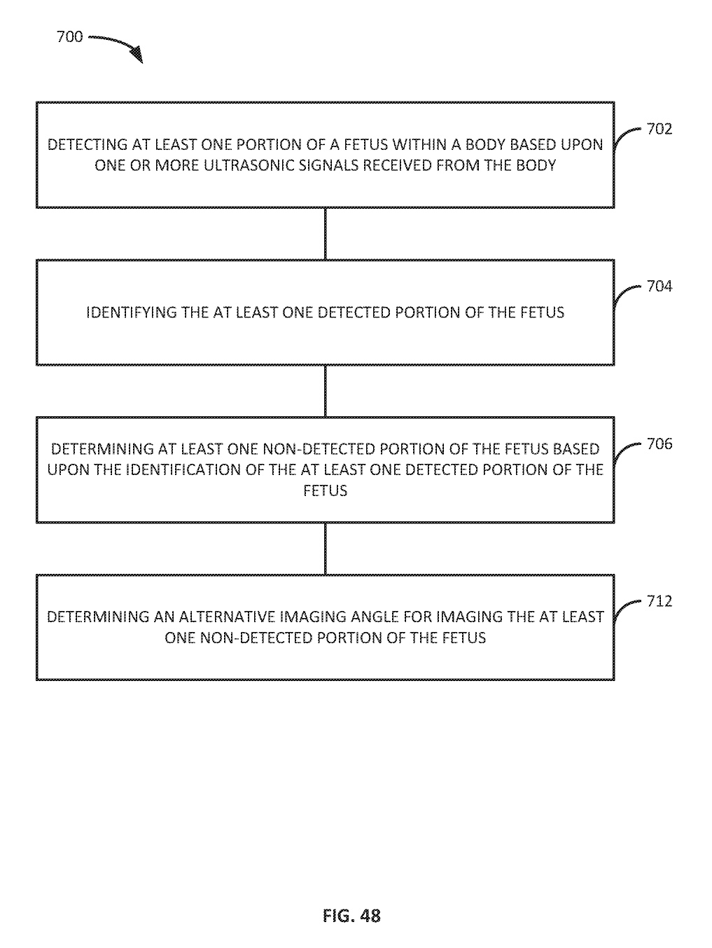

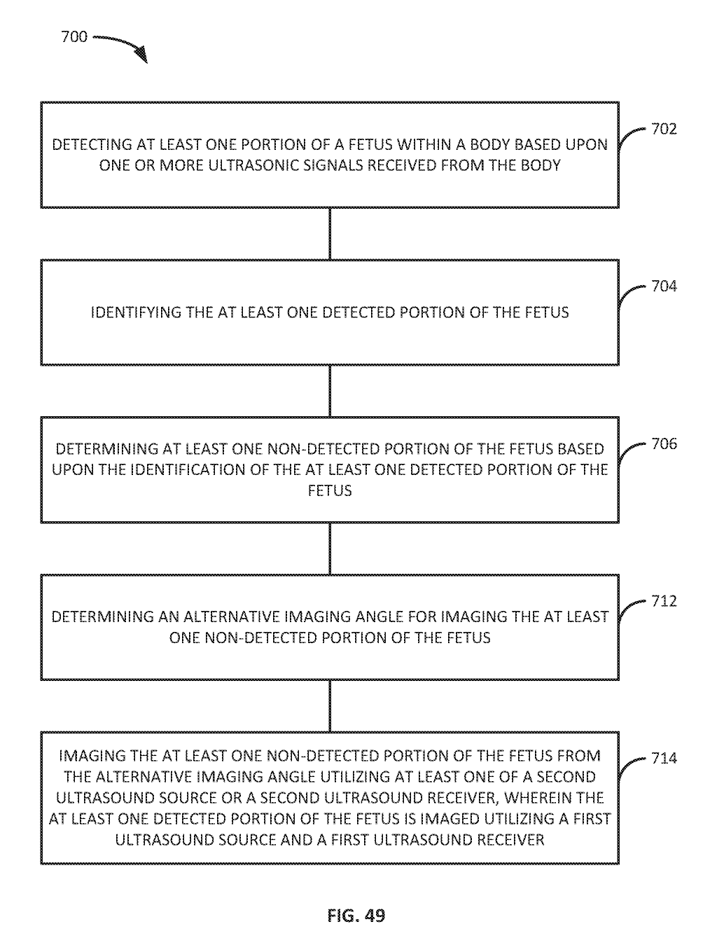

Referring to FIG. 48, an alternative imaging angle for imaging the non-detected portion of the fetus can be determined (block 712). For example, an alternative imaging angle can be determined for imaging the non-detected portion. In some embodiments, an instruction can be provided for imaging the non-detected portion of the fetus from the alternative imaging angle. As shown in FIG. 49, the non-detected portion can be imaged from the alternative imaging angle utilizing a second ultrasound element, a second array, or a second combination of ultrasound elements (block 714).

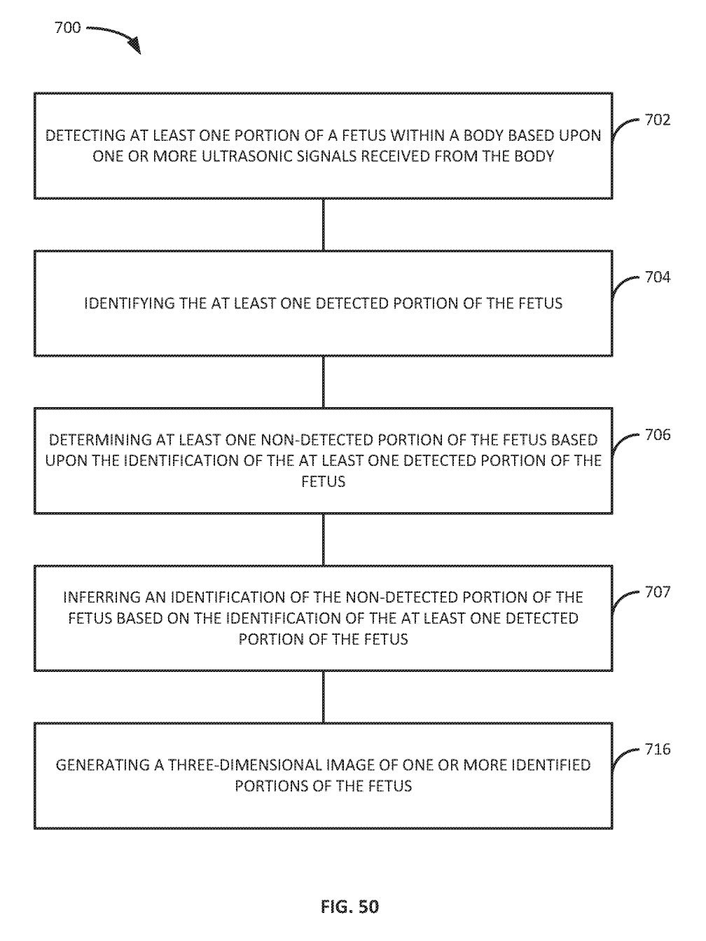

Referring to FIG. 50, a three-dimensional image can be generated based upon one or more identified portions of the fetus (block 716). For example, the controller can create a three-dimensional graphical rendering based upon the one or more detected portions of the fetus. The image can further include computer-generated or re-imaged renderings of the one or more non-detected portions of the fetus. For example, the controller can create a computer-generated rendering of at least one non-detected portion of the fetus based upon the one or more detected portions of the fetus.

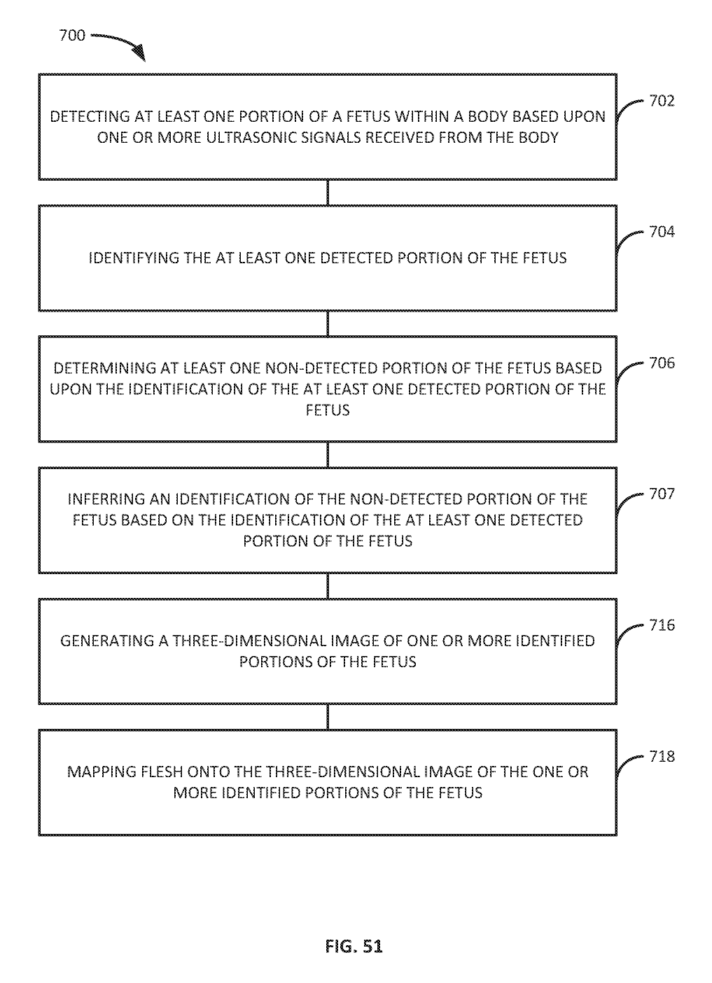

As shown in FIG. 51, the method 700 can further include mapping flesh onto the three-dimensional image of the one or more identified portions of the fetus (block 718). For example, the controller can apply a texture map to the three-dimensional graphical rendering of the fetus. By way of further example, the controller can apply a texture map based upon a predetermined texture map, a genomic element, a skin tone, a demographic, a stage of development, an image of a person, or the like.

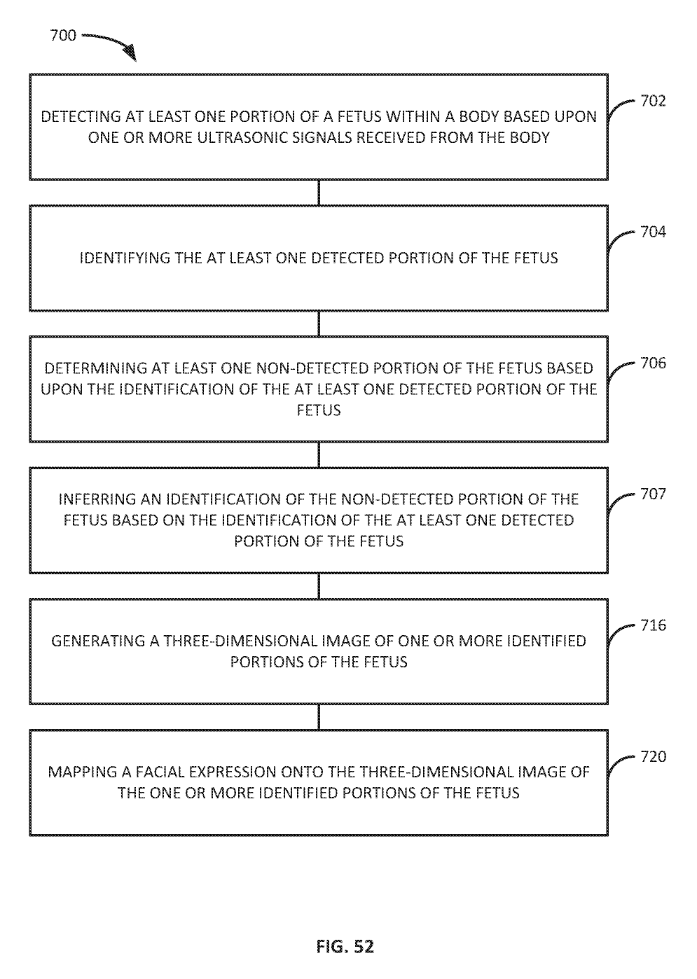

As shown in FIG. 52, the method 700 can further include mapping a facial expression onto the three-dimensional image of the one or more identified portions of the fetus (block 720). For example, the controller can map a facial expression onto the three-dimensional graphical rendering of the fetus. By way of further example, the controller can map a facial expression that is randomly generated or based upon a time, a date, a detected sound, a detected motion, a detected temperature, an identified fetal posture, a user input, or the like.

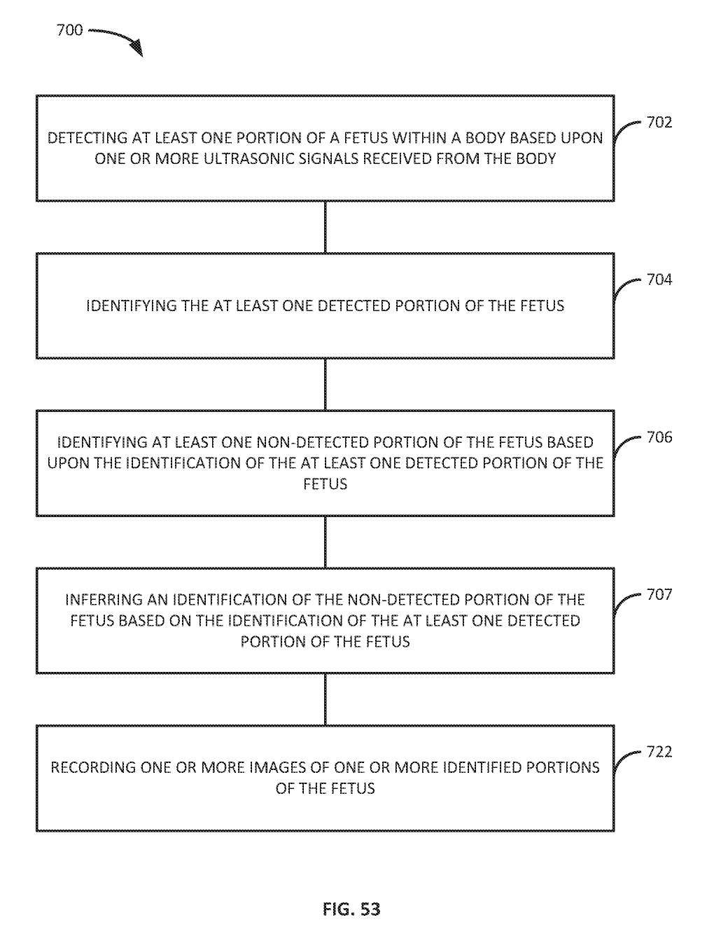

Referring to FIG. 53, one or more images (e.g., ultrasound images or computer-generated images) of the one or more portions of the fetus can be generated or recorded (block 722). For example, the controller can generate or record an image based upon the one or more received ultrasonic signals. In some embodiments, the one or more images are generated or recorded periodically, according to a schedule, or in response to a user input, a request from a second device, or detection of fetal activity above or below a threshold fetal activity. In some embodiments, the one or more recorded images of the one or more portions of the fetus are stored in at least one of a local storage device, a remote storage device, a network of storage devices, or a cloud-based storage network. For example, the controller can store the one or more recorded images to a storage device (e.g., storage device 136).

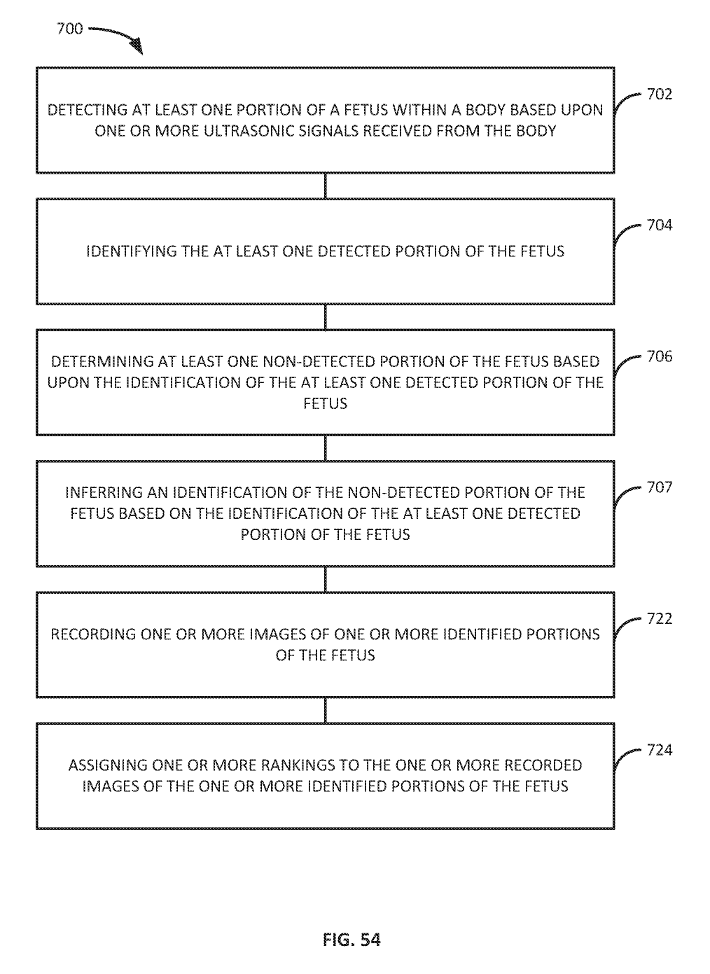

As shown in FIG. 54, the method 700 can further include assigning one or more rankings to the one or more recorded images of the one or more portions of the fetus (block 724). For example, the controller can assign a score or ranking to a recorded image of the fetus based upon an image quality or other characteristic of the image.

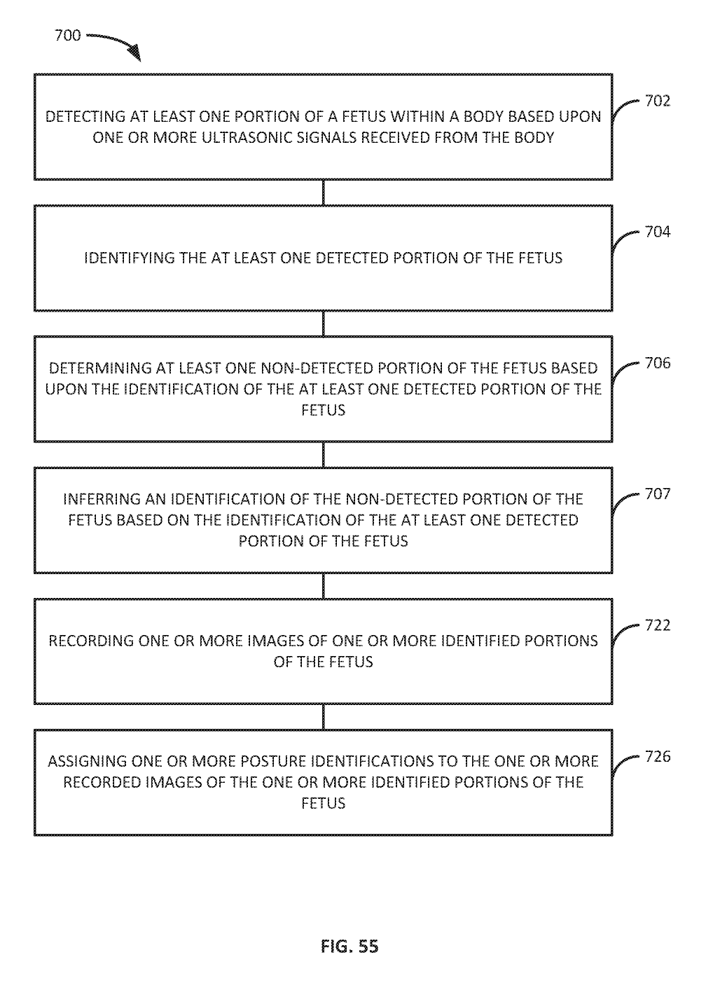

As shown in FIG. 55, the method 700 can further include assigning one or more posture identifications to the one or more recorded images of the one or more portions of the fetus (block 726). For example, the controller can identify a posture of the fetus based upon connectivity data associated with the one or more identified portions of the fetus or based upon a comparison between the recorded image and a previously stored image having an identified posture.

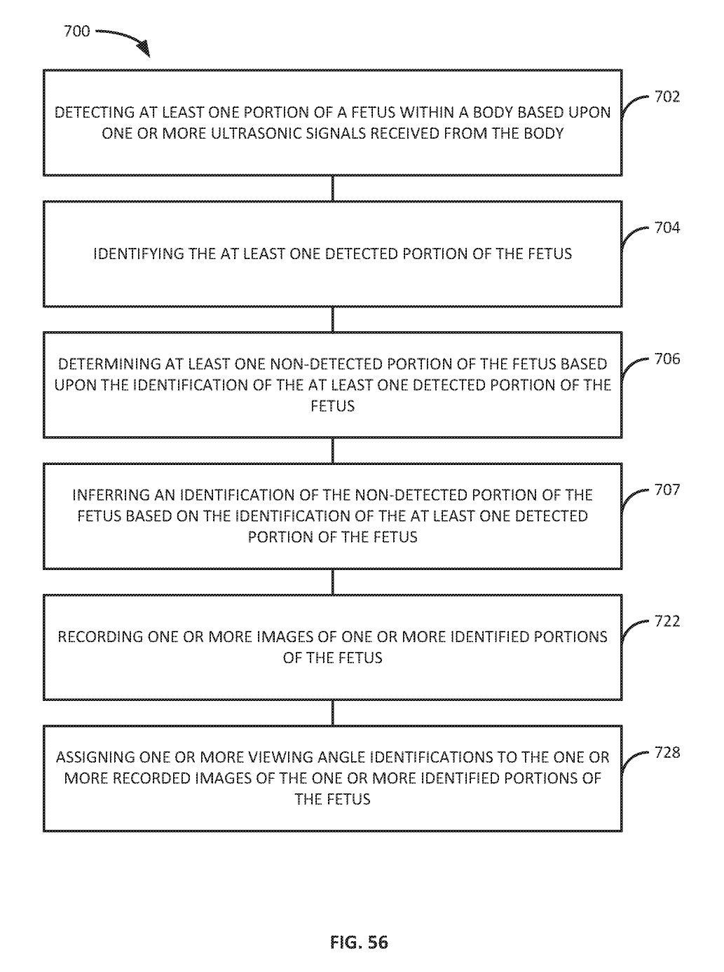

As shown in FIG. 56, the method 700 can further include assigning one or more viewing angle identifications to the one or more recorded images of the one or more portions of the fetus (block 728). For example, the controller can assign a viewing angle to an image of the fetus based upon a predetermined viewing angle associated with an ultrasound element, or the viewing angle can be derived from the image of the fetus.

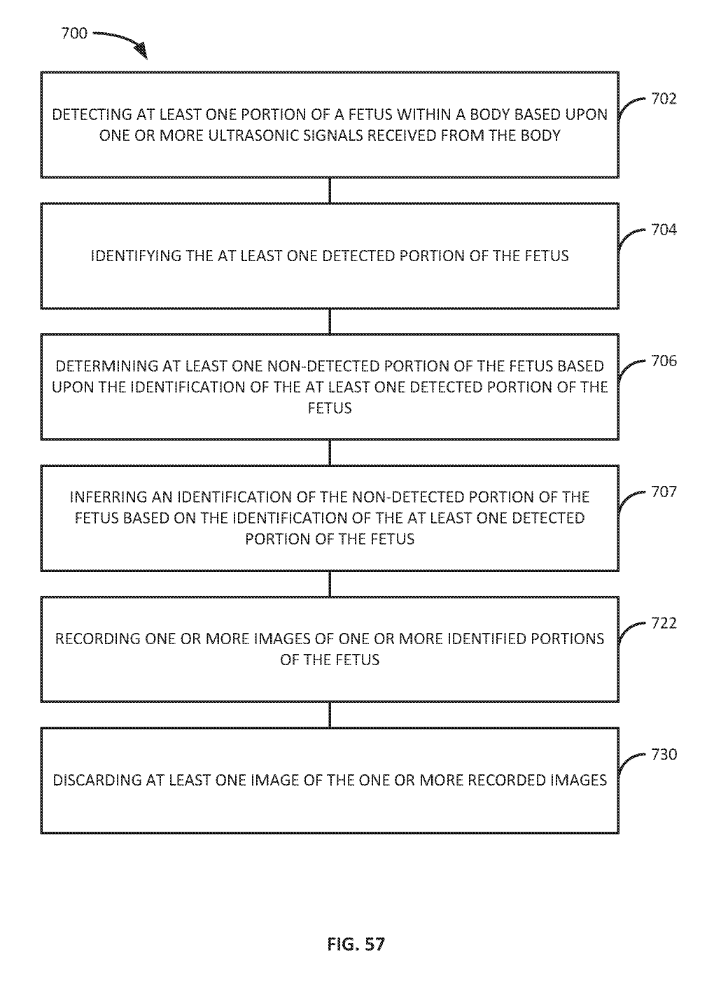

As shown in FIG. 57, the method 700 can further include discarding at least one image of the one or more recorded images (block 730). For example, the controller can delete an image from the storage device to increase available storage capacity or remove undesirable images. In some embodiments, an image can be discarded based upon detecting at least one of a redundant viewing angle, a redundant fetal posture, an obscured portion of the fetus, or an expired timestamp.

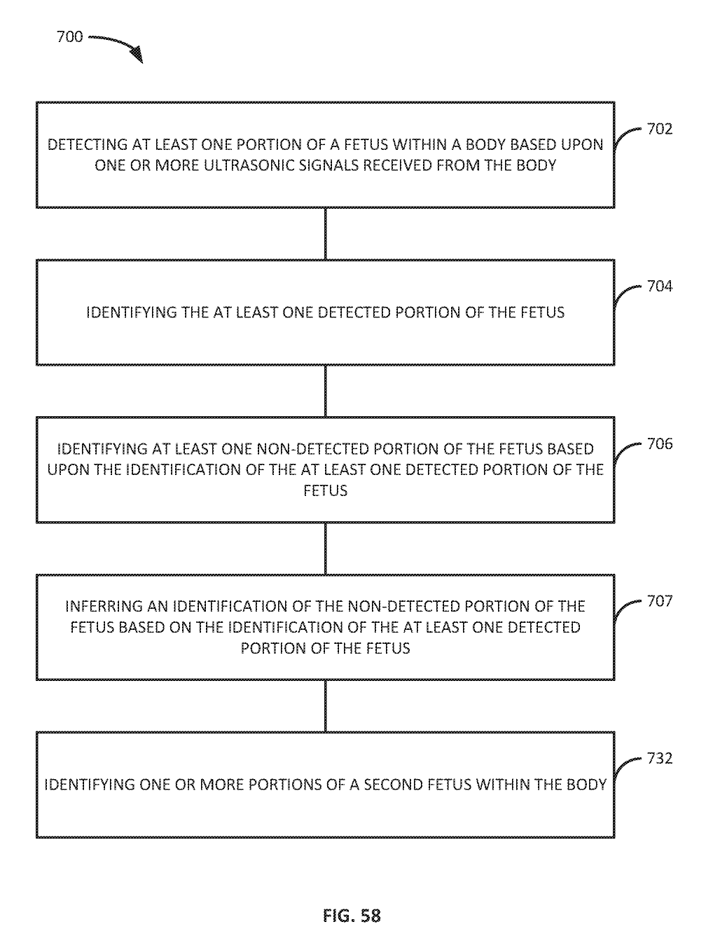

Referring to FIG. 58, one or more portions of a second fetus within the body can be identified (block 732). For example, the controller can identify one or more portions of the second fetus based upon the one or more received ultrasonic signals. In some embodiments, one or more operations described herein for the first fetus can be performed for the second fetus. In some embodiments, identification of one or more portions of the second fetus or determination of positioning or connectivity information can be based upon the first fetus. For example, the controller can identify one or more portions of the second fetus based upon one or more identified portions of the first fetus.

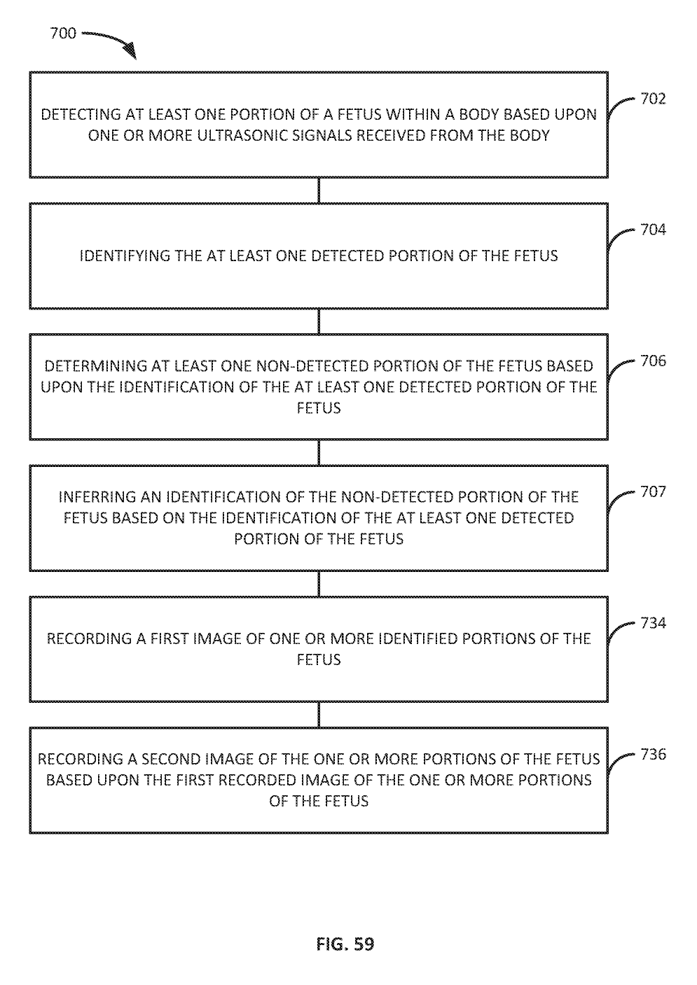

An embodiment of method 700 is shown in FIG. 59 to include recording a first image of the one or more portions of the fetus (block 734) and recording a second image of the one or more portions of the fetus based upon the first recorded image of the one or more portions of the fetus (block 736). For example, a second image can be recorded or collected because of an identified posture or viewing angle associated with the first image or lack thereof. In some embodiments, the second image can be recorded or collected because the first image includes a non-identifiable or obscured portion of the fetus. In some embodiments, the second image can be recorded or collected because of a score or ranking (e.g., computer-generated or user-driven score or ranking) assigned to the first image.

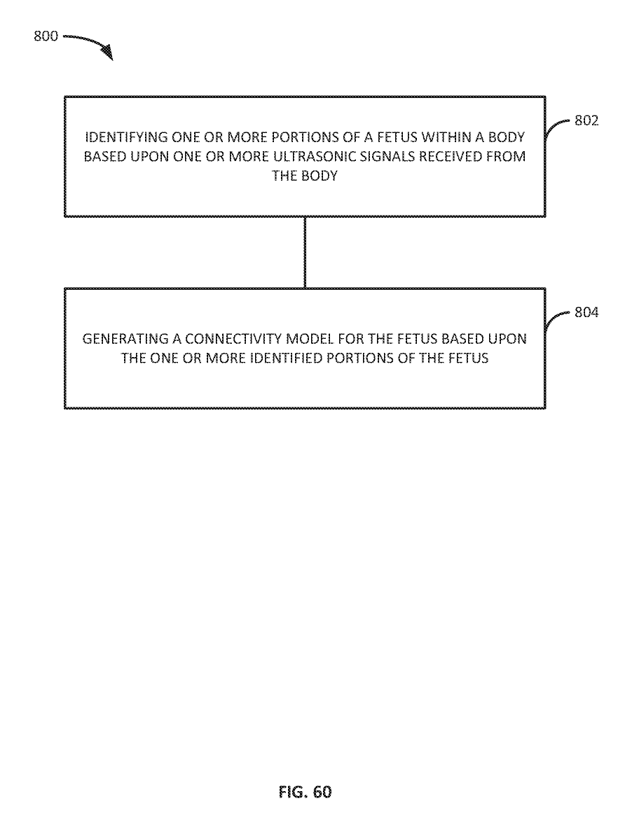

FIGS. 60-62 show various embodiments of a method 800 of providing a connectivity model for an ultrasonically imaged fetus. The method 800 can be performed by one or more components of the fetal imaging device 100 described herein or any other device (e.g., remote device 400) configured to support the operations described herein. At block 802, one or more portions of a fetus within a body can be identified based upon one or more ultrasonic signals received from the body. For example, the one or more received ultrasonic signals can be received by at least one ultrasound element (e.g., ultrasound element 102) configured for ultrasonography or shear wave elastography, and a controller (e.g., controller 106) can identify the one or more portions of the fetus. The one or more portions can simply be identified as belonging to the fetus or can be further identified as including one or more particular anatomical structures of the fetus. At block 804, a connectivity model can be generated for the fetus based upon the one or more identified portions of the fetus. For example, the controller can generate a connectivity model for the fetus based upon the one or more identified portions of the fetus.

The generated connectivity model can include a predetermined connectivity of a first portion of the fetus and a second portion of the fetus. In some embodiments, the first portion of the fetus includes an identified portion of the fetus, and the second portion of the fetus includes a non-detected portion of the fetus. In some embodiments, the connectivity model can include at least one of hard tissue connectivity model or a soft tissue connectivity model. In some embodiments, the connectivity model can be based upon a detected spatial parameter (e.g., length or width) associated with the one or more identified portions of the fetus, a development stage, a gender, or any other detected or specified information about the fetus. In some embodiments, the connectivity model is based upon an identified posture of the fetus. For example, the posture can be identified utilizing image recognition, based upon at least one of a determined position or a determined orientation of at least one identified portion of the fetus, or based upon an identified connectivity of a first portion of the fetus and a second portion of the fetus. In some embodiments, the identified posture is selected from a group of predetermined postures of the fetus. For example, the identified posture can be selected based upon a comparison between a collected image of the fetus and another image that is associated with an identified posture. In some embodiments, the connectivity model is based upon an anatomical model that characterizes at least one of appearance, relative size attributes, or connectivity of fetal tissue structures.

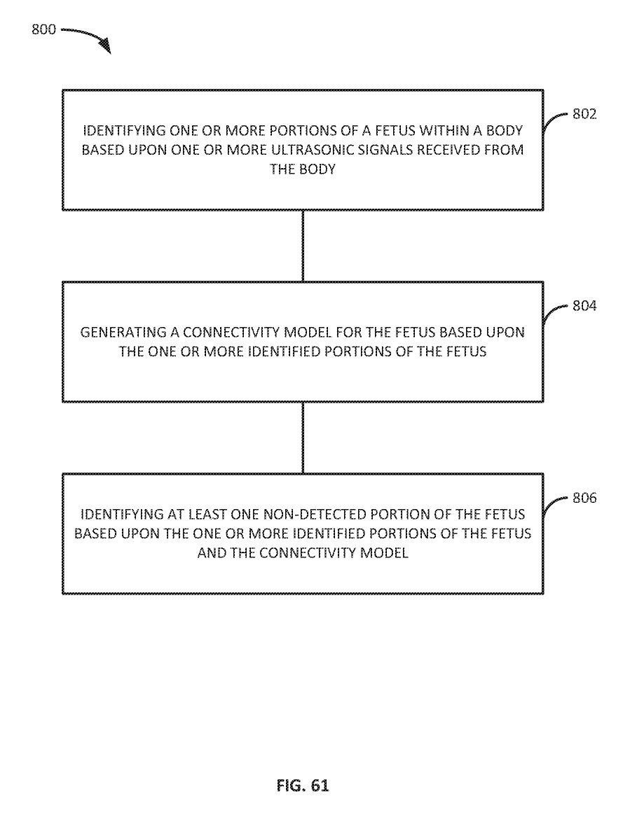

As shown in FIG. 61, the method can also include identifying at least one non-detected portion of the fetus based upon the one or more identified portions of the fetus and the connectivity model (block 806). For example, a non-detected portion of the fetus can be identified based upon connectivity information derived from the connectivity model that includes a connectivity between the one or more identified portions of the fetus and the non-detected portion of the fetus.

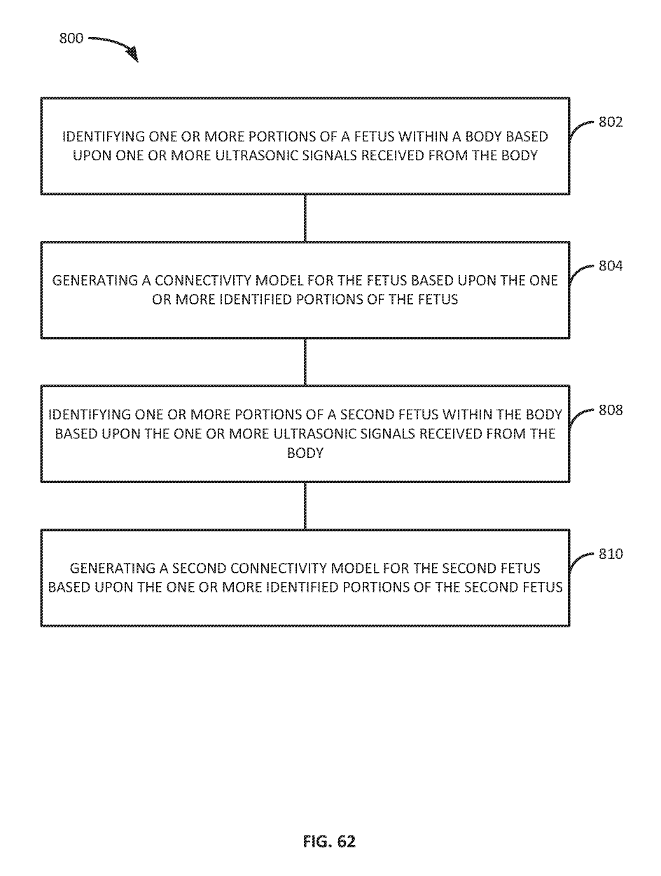

As shown in FIG. 62, the method can also include identifying one or more portions of a second fetus within the body based upon the one or more ultrasonic signals received from the body (block 808), and generating a second connectivity model for the second fetus based upon the one or more identified portions of the second fetus (block 810). This can be the same connectivity model as generated for the first fetus or a second connectivity model based upon one or more attributes of the second fetus. For example, the second connectivity model can be based upon a detected spatial parameter (e.g., length or width) associated with the one or more identified portions of the second fetus, a development stage, a gender, or any other detected or specified information about the second fetus. In some embodiments, the second connectivity model is integrated with the first connectivity model in a combined connectivity model that can include positioning information of one or more portions of a first fetus relative to one or more portions of a second fetus within a body. In some embodiments, the second connectivity model is based upon the first connectivity model or influenced by one or more attributes of the first fetus. For example, the second connectivity model can be at least partially based on relative size attributes, relative orientations, or relative positions of the one or more identified portions of the first fetus and the one or more identified portions of the second fetus.

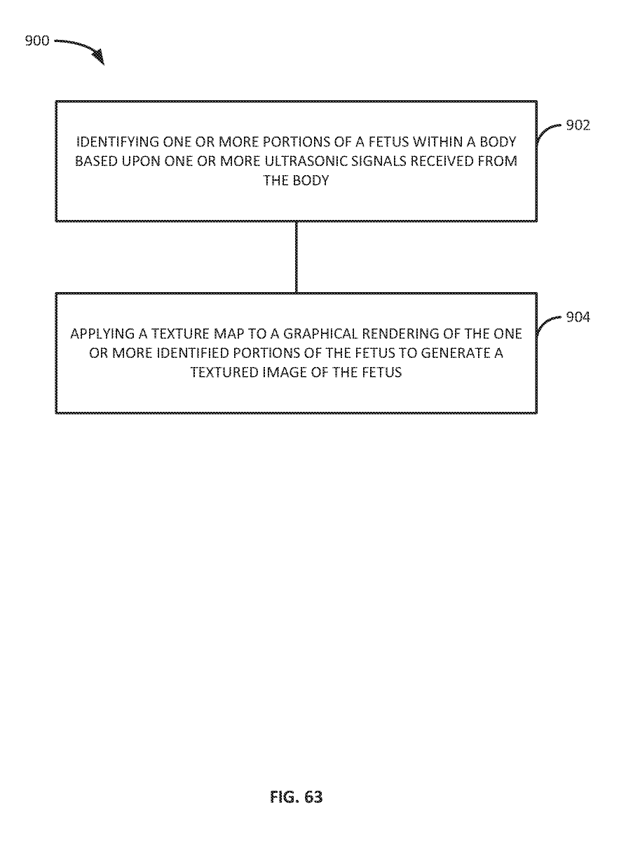

FIGS. 63-65 show various embodiments of a method 900 of providing a textured image of an ultrasonically imaged fetus. The method 900 can be performed by one or more components of the fetal imaging device 100 described herein or any other device (e.g., remote device 400) configured to support the operations described herein. At block 902, one or more portions of a fetus within a body can be identified based upon one or more ultrasonic signals received from the body. For example, the one or more received ultrasonic signals can be received by at least one ultrasound element (e.g., ultrasound element 102) configured for ultrasonography or shear wave elastography, and a controller (e.g., controller 106) can identify the one or more portions of the fetus. The one or more portions can simply be identified as belonging to the fetus or can be further identified as including one or more particular anatomical structures of the fetus. At block 904, a texture map can be applied to a graphical rendering of the one or more identified portions of the fetus to generate a textured image of the fetus. For example, the controller can apply a texture map to a two-dimensional or three-dimensional graphical rendering of the one or more identified portions of the fetus to generate a textured image with flesh mapped onto the one or more identified portions of the fetus. In some embodiments, the textured image can include a three-dimensional image of the fetus. In some embodiments, the textured image can be viewed from multiple viewing angles via an interface device (e.g., interface device 138). In some embodiments, the texture map includes a predetermined texture map. In some embodiments, the texture map is based on a specified or detected genomic element, skin tone, demographic, gender, or development stage associated with the fetus. In some embodiments, the texture map is at least partially based on an image of a person. For example, the texture map can be based upon an image of at least one relative, such as a parent, sibling, uncle, aunt, cousin, grandparent, or the like.

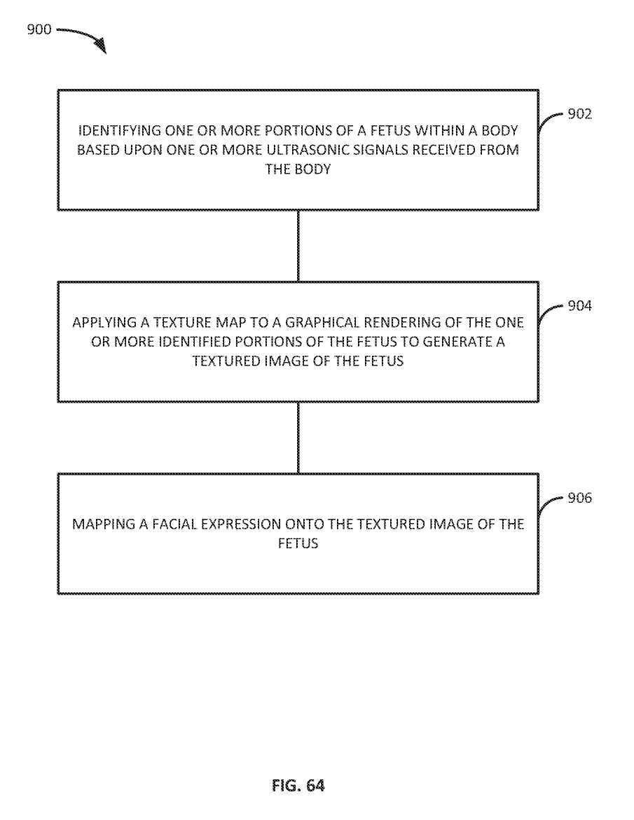

As shown in FIG. 64, the method 900 can also include mapping a facial expression onto the textured image of the one or more identified portions of the fetus (block 906). For example, the controller can map a facial expression onto a textured three-dimensional graphical rendering of the fetus. In some embodiments, the facial expression can be randomly generated or based upon a time, a date, a detected sound, a detected motion, a detected temperature, an identified fetal posture, a user input, or the like.

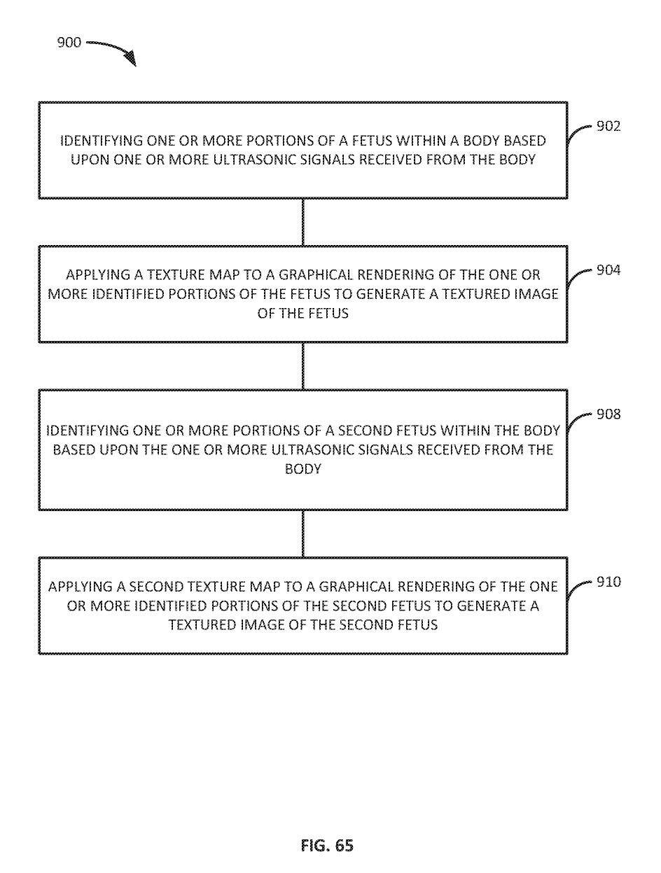

As shown in FIG. 65, the method can also include identifying one or more portions of a second fetus within the body based upon the one or more ultrasonic signals received from the body (block 908), and applying a second texture map to a graphical rendering of the one or more identified portions of the second fetus to generate a textured image of the second fetus (block 910). This can be the same texture map as applied to the graphical rendering of the one or more portions of the first fetus or a second texture map that is different from the first texture map. For example, the second texture map can be based upon one or more attributes of the second fetus. By way of further example, the second texture map can be associated with a different gender or different relative. In some embodiments, the second texture map is based upon the first texture map. For example, the second texture map can include a modified version of the first texture map (e.g., modified for a different gender).

This disclosure has been made with reference to various example embodiments. However, changes and modifications can be made to the embodiments without departing from the scope of the present disclosure. For example, various operational steps, as well as components for carrying out operational steps, can be implemented in alternate ways depending upon the particular application or in consideration of any number of cost functions associated with the operation of the system; e.g., one or more of the steps can be deleted, modified, or combined with other steps.

Additionally, principles of the present disclosure, including components, can be reflected in a computer program product on a computer-readable storage medium having computer-readable program code means embodied in the storage medium. Any tangible, non-transitory computer-readable storage medium can be utilized, including magnetic storage devices (hard disks, floppy disks, and the like), optical storage devices (CD-ROMs, DVDs, Blu-ray discs, and the like), flash memory, and/or the like. These computer program instructions can be loaded onto a general purpose computer, special purpose computer, or other programmable data processing apparatus to produce a machine, such that the instructions that execute on the computer or other programmable data processing apparatus create a means for implementing the functions specified. These computer program instructions can also be stored in a computer-readable memory that can direct a computer or other programmable data processing apparatus to function in a particular manner, such that the instructions stored in the computer-readable memory produce an article of manufacture, including implementing means that implement the function specified. The computer program instructions can also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that can cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but can include other elements not expressly listed or inherent to such process, method, system, article, or apparatus.

In an embodiment, the system is integrated in such a manner that the system operates as a unique system configured specifically for function of the fetal imaging device, and any associated computing devices of the system operate as specific use computers for purposes of the claimed system, and not general use computers. In an embodiment, one or more associated computing devices of the system operate as specific use computers for purposes of the claimed system, and not general use computers. In an embodiment, at least one of the associated computing devices of the system is hardwired with a specific ROM to instruct the at least one computing device. In an embodiment, the fetal imaging device and system effects an improvement at least in the technological field of fetal imaging and/or monitoring.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer can opt for a mainly hardware and/or firmware vehicle; alternatively, if flexibility is paramount, the implementer can opt for a mainly software implementation; or, yet again alternatively, the implementer can opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Optical aspects of implementations will typically employ optically-oriented hardware, software, and or firmware.

In some implementations described herein, logic and similar implementations can include software or other control structures. Electronic circuitry, for example, can have one or more paths of electrical current constructed and arranged to implement various functions as described herein. In some implementations, one or more media can bear a device-detectable implementation when such media hold or transmit a device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations can include executing a special-purpose instruction sequence or otherwise invoking circuitry for enabling, triggering, coordinating, requesting, or otherwise causing one or more occurrences of any functional operations described above. In some variants, operational or other logical descriptions herein can be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression can be manifested as a Verilog-type hardware description or other circuitry model before physical implementation in hardware, especially for basic operations or timing-critical applications.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, and/or virtually any combination thereof and a wide range of components that can impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Examples of electro-mechanical systems include but are not limited to a variety of consumer electronics systems, medical devices, as well as other systems such as motorized transport systems, factory automation systems, security systems, and/or communication/computing systems. Electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

At least a portion of the systems and/or processes described herein can be integrated into an image processing system. A typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system can be implemented utilizing suitable commercially available components, such as those typically found in digital still systems and/or digital motion systems.

At least a portion of the systems and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system can be implemented utilizing suitable commercially available components, such as those typically found in data computing/communication and/or network computing/communication systems.

At least a portion of the systems and/or processes described herein can be integrated into a mote system. A typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system can be implemented utilizing suitable components, such as those found in mote computing/communication systems. Specific examples of such components entail such as Intel Corporation's and/or Crossbow Corporation's mote components and supporting hardware, software, and/or firmware.

The herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, based upon the teachings herein, changes and modifications can be made without departing from the subject matter described herein and its broader aspects and, therefore, the appended claims are to encompass within their scope all such changes and modifications as are within the true spirit and scope of the subject matter described herein.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method, comprising:
detecting at least one portion of a fetus within a body based upon one or more ultrasonic signals received from the body;
identifying the at least one detected portion of the fetus;
determining at least one non-detected portion of the fetus based upon the identification of the at least one detected portion of the fetus; and
inferring an identification of the non-detected portion of the fetus based on the identification of the at least one detected portion of the fetus.

2.-5. (canceled)

6. The method of claim 1, wherein inferring the identification of the non-detected portion of the fetus based on the identification of the at least one detected portion of the fetus includes:
identifying the at least one non-detected portion of the fetus with a connectivity model, the connectivity model including a predetermined connectivity of the at least one non-detected portion of the fetus and the at least one detected portion of the fetus.

7. (canceled)

8. (canceled)

9. The method of claim 1, further including:
determining a position of the at least one non-detected portion of the fetus based upon a position of at least one detected portion of the fetus and an anatomical model.

10. The method of claim 1, further including:
determining an orientation of the at least one non-detected portion of the fetus based upon an orientation of at least one detected portion of the fetus and an anatomical model.

11.-16. (canceled)

17. The method of claim 1, further including:
generating a three-dimensional image of one or more identified portions of the fetus.

18. The method of claim 17, further including:
mapping flesh onto the three-dimensional image of the one or more identified portions of the fetus.

19. The method of claim 18, wherein the flesh mapped onto the three-dimensional image of the one or more identified portions of the fetus is based on a predetermined texture map.

20. The method of claim 19, wherein the texture map is at least partially based on at least one of a genomic element, a skin tone, a demographic, a stage of development, or an image of a person.

21. (canceled)

22. (canceled)

23. The method of claim 1, further including:
recording one or more images of one or more identified portions of the fetus.

24. The method of claim 23, further including:
assigning one or more rankings to the one or more recorded images of the one or more identified portions of the fetus.

25. The method of claim 23, further including:
assigning one or more posture identifications to the one or more recorded images of the one or more identified portions of the fetus.

26. The method of claim 23, further including:
assigning one or more viewing angle identifications to the one or more recorded images of the one or more identified portions of the fetus.

27. The method of claim 23, wherein recording the one or more images of the one or more portions of the fetus includes:
recording the one or more images of the one or more identified portions of the fetus based on at least one of a user input, a request received from a second device, a schedule, detecting fetal activity above a threshold fetal activity, or detecting fetal activity below a threshold fetal activity.

28.-34. (canceled)

35. The method of claim 1, further including:
recording a first image of one or more identified portions of the fetus;
recording a second image of the one or more portions of the fetus based upon the first recorded image of the one or more portions of the fetus.

36. The method of claim 35, wherein recording the second image of the one or more portions of the fetus based upon the first recorded image of the one or more portions of the fetus includes:
recording the second image of the one or more portions of the fetus in response to at least one of the first recorded image being associated with an identified posture of the fetus, the first recorded image being associated with an identified viewing angle of the fetus, the first recorded image being associated with non-identifiable posture of the fetus, or the first recorded image including a non-identifiable portion of the fetus.

37. The method of claim 35, wherein recording the second image of the one or more portions of the fetus based upon the first recorded image of the one or more portions of the fetus includes:
recording the second image of the one or more portions of the fetus based upon at least one of a user-driven score or a user-driven ranking of the first image.

38. A non-transitory computer readable medium with one or more executable instructions stored thereon, comprising one or more instructions that cause at least one computer processor executing the one or more instructions to:
detect at least one portion of a fetus within a body based upon one or more ultrasonic signals received from the body;
identify the at least one detected portion of the fetus;
determine at least one non-detected portion of the fetus based upon the identification of the at least one detected portion of the fetus; and
infer an identification of the non-detected portion of the fetus based on the identification of the at least one detected portion of the fetus.

39.-42. (canceled)

43. The non-transitory computer readable medium of claim 38, wherein the one or more instructions that cause the at least one computer processor to infer the identification of the non-detected portion of the fetus based on the identification of the at least one detected portion of the fetus include one or more instructions that cause the at least one computer processor to:
identify the at least one non-detected portion of the fetus with a connectivity model, the connectivity model including a predetermined connectivity of the at least one non-detected portion of the fetus and the at least one detected portion of the fetus.

44.-47. (canceled)

48. The non-transitory computer readable medium of claim 38, further including one or more instructions that cause the at least one computer processor to:
determine a position of the at least one non-detected portion of the fetus based upon a position of at least one detected portion of the fetus and a connectivity model, the connectivity model including a predetermined connectivity of the at least one non-detected portion of the fetus and the at least one detected portion of the fetus.

49. The non-transitory computer readable medium of claim 38, further including one or more instructions that cause the at least one computer processor to:
determine an orientation of the at least one non-detected portion of the fetus based upon an orientation of at least one detected portion of the fetus and a connectivity model, the connectivity model including a predetermined connectivity of the at least one non-detected portion of the fetus and the at least one detected portion of the fetus.

50.-53. (canceled)

54. The non-transitory computer readable medium of claim 38, further including one or more instructions that cause the at least one computer processor to:
generate a three-dimensional image of one or more identified portions of the fetus.

55. The non-transitory computer readable medium of claim 54, further including one or more instructions that cause the at least one computer processor to:
map flesh onto the three-dimensional image of the one or more identified portions of the fetus.

56. The non-transitory computer readable medium of claim 55, wherein the flesh mapped onto the three-dimensional image of the one or more identified portions of the fetus is based on a predetermined texture map.

57. The non-transitory computer readable medium of claim 56, wherein the texture map is at least partially based on at least one of a genomic element, a skin tone, a demographic, a stage of development, or an image of a person.

58.-74. (canceled)

75. A method, comprising:
identifying one or more portions of a fetus within a body based upon one or more ultrasonic signals received from the body; and
generating a connectivity model for the fetus based upon the one or more identified portions of the fetus.

76.-78. (canceled)

79. The method of claim 75, wherein the connectivity model is based upon an identified connectivity of a first portion of the fetus and a second portion of the fetus.

80. The method of claim 75, wherein the connectivity model is based upon a predetermined connectivity of a first portion of the fetus and a second portion of the fetus.

81. The method of claim 75, wherein the connectivity model is based upon an identified posture of the fetus.

82. The method of claim 81, wherein the identified posture of the fetus is selected from a group of predetermined postures of the fetus.

83. The method of claim 82, wherein the identified posture of the fetus is selected based upon an identified connectivity of a first portion of the fetus and a second portion of the fetus.

84.-120. (canceled)
